SEQUENCE LISTING (45 sequences)

SEQ ID NO: 1 - protein, 119 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARSTLWFSEFDYWGQGTLVTVSS

SEQ ID NO: 2 - protein, 110 aa, Artificial Sequence
  Description of Artificial Sequence Synthetic polypeptide
QSVLTQPSSVSASPGQSITISCSGTSSDVGGHNYVSWYQQHPGKAPKLMIYDVTKRPSGVPDRFSGSKSGNTASLTVSGLQAEDEADYYCLSYAGSRVYVFGTGTKLTVL

SEQ ID NO: 3 - protein, 444 aa, Artificial Sequence
  Description of Artificial Sequence Synthetic polypeptide
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 219 aa, Artificial Sequence
  Description of Artificial Sequence Synthetic polypeptide
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 6 - protein, 451 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 213 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 448 aa, Artificial Sequence
  Description of Artificial Sequence Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 219 aa, Artificial Sequence
  Description of Artificial Sequence Synthetic polypeptide
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 222 aa, Artificial Sequence
  Description of Artificial Sequence Synthetic polypeptide
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPGSSKSTSGTAALGCLVKDYFPEPVTVSWNSGALTSGVHTFPAVLQSSGLYSLSSVVTVPSSSLGTQTYICNVNHKPSNTKVDKKVEP

SEQ ID NO: 11 - protein, 211 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 12 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 13 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 14 - protein, 300 aa, Macaca fascicularis
MANCEFSPVSGDKPCCRLSRRAQVCLGVCLLVLLILVVVVAVVLPRWRQQWSGSGTTSRFPETVLARCVKYTEVHPEMRHVDCQSVWDAFKGAFISKYPCNITEEDYQPLVKLGTQTVPCNKTLLWSRIKDLAHQFTQVQRDMFTLEDMLLGYLADDLTWCGEFNTFEINYQSCPDWRKDCSNNPVSVFWKTVSRRFAETACGVVHVMLNGSRSKIFDKNSTFGSVEVHNLQPEKVQALEAWVIHGGREDSRDLCQDPTIKELESIISKRNIRFFCKNIYRPDKFLQCVKNPEDSSCLSG

SEQ ID NO: 15 - protein, 304 aa, Mus musculus
MANYEFSQVSGDRPGCRLSRKAQIGLGVGLLVLIALVVGIVVILLRPRSLLVWTGEPTTKHFSDIFLGRCLIYTQILRPEMRDQNCQEILSTFKGAFVSKNPCNITREDYAPLVKLVTQTIPCNKTLFWSKSKHLAHQYTWIQGKMFTLEDTLLGYIADDLRWCGDPSTSDMNYVSCPHWSENCPNNPITVFWKVISQKFAEDACGVVQVMLNGSLREPFYKNSTFGSVEVFSLDPNKVHKLQAWVMHDIEGASSNACSSSSLNELKMIVQKRNMIFACVDNYRPARFLQCVKNPEHPSCRLNT

SEQ ID NO: 16 - protein, 452 aa, Artificial Sequence
  Description of Artificial Sequence Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 118 aa, Artificial Sequence
  Description of Artificial Sequence Synthetic polypeptide
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSS

SEQ ID NO: 19 - protein, 110 aa, Artificial Sequence
  Description of Artificial Sequence Synthetic polypeptide
QAGLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVL

SEQ ID NO: 20 - protein, 118 aa, Artificial Sequence
  Description of Artificial Sequence Synthetic polypeptide
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWSGEFTDWGQGTLVTVSS

SEQ ID NO: 21 - protein, 110 aa, Artificial Sequence
  Description of Artificial Sequence Synthetic polypeptide
QAGLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL

SEQ ID NO: 22 - protein, 118 aa, Artificial Sequence
  Description of Artificial Sequence Synthetic polypeptide
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWGGEFTNWGQGTLVTVSS

SEQ ID NO: 23 - protein, 110 aa, Artificial Sequence; identical to SEQ ID NO: 21
  Description of Artificial Sequence Synthetic polypeptide

SEQ ID NO: 24 - protein, 118 aa, Artificial Sequence
  Description of Artificial Sequence Synthetic polypeptide
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWGGEFTDWGQGTLVTVSS

SEQ ID NO: 25 - protein, 110 aa, Artificial Sequence; identical to SEQ ID NO: 21
  Description of Artificial Sequence Synthetic polypeptide

SEQ ID NO: 26 - protein, 118 aa, Artificial Sequence
  Description of Artificial Sequence Synthetic polypeptide
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWSGEFTDWGQGTLVTVSS

SEQ ID NO: 27 - protein, 110 aa, Artificial Sequence; identical to SEQ ID NO: 19
  Description of Artificial Sequence Synthetic polypeptide

SEQ ID NO: 28 - protein, 118 aa, Artificial Sequence
  Description of Artificial Sequence Synthetic polypeptide
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWGGEFTDWGQGTLVTVSS

SEQ ID NO: 29 - protein, 110 aa, Artificial Sequence; identical to SEQ ID NO: 19
  Description of Artificial Sequence Synthetic polypeptide

SEQ ID NO: 30 - protein, 118 aa, Artificial Sequence; identical to SEQ ID NO: 20
  Description of Artificial Sequence Synthetic polypeptide

SEQ ID NO: 31 - protein, 110 aa, Artificial Sequence; identical to SEQ ID NO: 19
  Description of Artificial Sequence Synthetic polypeptide

SEQ ID NO: 32 - protein, 118 aa, Artificial Sequence
  Description of Artificial Sequence Synthetic polypeptide
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGQGTLVTVSS

SEQ ID NO: 33 - protein, 110 aa, Artificial Sequence
  Description of Artificial Sequence Synthetic polypeptide
QSVLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL

SEQ ID NO: 34 - protein, 118 aa, Artificial Sequence
  Description of Artificial Sequence Synthetic polypeptide
QVQLVESGGGLVKPGGSLRLSCAASGFTFSDDYMSWIRQAPGKGLEWVASVSSGRPTTYYADSVRGRFTISRDNAKNSLYLQMNSLRAEDTAVYYCAREDWGGEFTDWGQGTLVTVSS

SEQ ID NO: 35 - protein, 110 aa, Artificial Sequence; identical to SEQ ID NO: 33
  Description of Artificial Sequence Synthetic polypeptide

SEQ ID NO: 36 - protein, 118 aa, Artificial Sequence; identical to SEQ ID NO: 18
  Description of Artificial Sequence Synthetic polypeptide

SEQ ID NO: 37 - protein, 110 aa, Artificial Sequence; identical to SEQ ID NO: 21
  Description of Artificial Sequence Synthetic polypeptide

SEQ ID NO: 38 - protein, 118 aa, Artificial Sequence; identical to SEQ ID NO: 20
  Description of Artificial Sequence Synthetic polypeptide

SEQ ID NO: 39 - protein, 110 aa, Artificial Sequence; identical to SEQ ID NO: 33
  Description of Artificial Sequence Synthetic polypeptide

SEQ ID NO: 40 - protein, 19 aa, Mus sp.
MEWSWVFLFFLSVTTGVHS

SEQ ID NO: 41 - protein, 10 aa, Artificial Sequence
  Description of Artificial Sequence Synthetic peptide
EPKSCDKTHT

SEQ ID NO: 42 - protein, 9 aa, Artificial Sequence
  Description of Artificial Sequence Synthetic peptide
PAPELLGGP

SEQ ID NO: 43 - protein, 12 aa, Artificial Sequence
  Description of Artificial Sequence Synthetic peptide
SVFLFPPKPKDT

SEQ ID NO: 44 - protein, 23 aa, Artificial Sequence
  Description of Artificial Sequence Synthetic peptide
EPKSCDKTHTCPPCPAPELLGGP

SEQ ID NO: 45 - protein, 303 aa, Rattus norvegicus
MANYEFSQVSEDRPGCRLTRKAQIGLGVGLLLLVALVVVVVIVLWPRSPLVWKGKPTTKHFADIILGRCLIYTQILRPEMRDQDCKKILSTFKRGFISKNPCNITNEDYAPLVKLVTQTIPCNKTLFWSKSKHLAHQYTWIQGKMFTLEDTLLGYIADDLRWCGDPSTSDMNYDSCPHWSENCPNNPVAVFWNVISQKFAEDACGVVQVMLNGSLSEPFYRNSTFGSVEVFNLDPNKVHKLQAWVMHDIKGTSSNACSSPSINELKSIVNKRNMIFACQDNYRPVRFLQCVKNPEHPSCRLNV

(19) United States
(12) Patent Application Publication
(10) Pub. No.: US 2021/0221886 A1
(43) Pub. Date: Jul. 22, 2021

(54) Compositions and Methods Comprising an Anti-CD47 Antibody in Combination with a Tumor Targeting Antibody

(71) Applicant: Sorrento Therapeutics, Inc., San Diego, CA (US)

(72) Inventors: Damien Bresson, San Diego, CA (US); Heyue Zhou, San Diego, CA (US); Christophe Pedros, San Diego, CA (US)

(21) Appl. No.: 17/112,587

(22) Filed: Dec. 4, 2020

Related U.S. Application Data
(60) Provisional application No. 62/943,926, filed on Dec. 5, 2019.
(60) Provisional application No. 63/030,464, filed on May 27, 2020.
(60) Provisional application No. 63/065,927, filed on Aug. 14, 2020.

(51) Int. Cl. C07K 16/28; A61P 35/02; A61K 9/00

(52) U.S. Cl. CPC C07K 16/2803; C07K 16/2887; C07K 16/2896; A61K 2039/507; A61K 9/0019; A61K 9/0085; C07K 2317/76; A61P 35/02

(57) ABSTRACT

The present disclosure provides compositions and methods comprising a first antibody comprising a fully human anti-CD47 antibody and a second antibody comprising an Fc portion that binds an Fcγ receptor on an effector cell. In various embodiments the anti-CD47 antibody used in the methods and compositions exhibits a low level of binding to red blood cells and does not induce hemagglutination even at high concentrations of antibody. In some embodiments, the second antibody comprises a tumor-targeting antibody including an antibody that binds CD20, PD-L1, CD38 or SLAMF7 antigens. The combination of the fully human anti-CD47 antibody and the second antibody can decrease cancer burden in a subject.

### RELATED APPLICATIONS

This application claims the benefit of U.S. Provisional Patent Application No. 62/943,926 filed Dec. 5, 2019; U.S. Provisional Patent Application No. 63/030,464 filed May 27, 2020; and U.S. Provisional Patent Application No. 63/065,927 filed Aug. 14, 2020, the contents of which are herein incorporated by reference in their entirety.

### INTRODUCTION

CD47 is a cell surface antigen overexpressed on many tumor cells. CD47 can inhibit phagocytosis by innate immune cells such as macrophages by engaging its receptor, signal regulatory protein alpha (SIRPα), on the surface of the immune cells. (Because it inhibits phagocytosis, CD47 is sometimes referred to as the “don't eat me” molecule.) Administration of anti-CD47 antibodies can relieve the inhibition of the native immune system by blocking the CD47-SIRPα interaction and thus provides an anticancer strategy.

In addition to being overexpressed on many tumor cells, CD47 is also expressed on some normal cells, including platelets and erythrocytes. Treatment of patients with anti-CD47 antibodies therefore can result in toxic effects to the patient resulting from normal blood cell binding. For example, the Phase I trial of anti-CD47 monoclonal antibody Hu5F9 (magrolimab) resulted in 57% of the treated patients experiencing transient anemia and 36% exhibiting hemagglutination of peripheral blood cells (Sikic et al. (2019) J. Clinical Oncol. 37:946-953).

### SUMMARY

The present disclosure provides compositions and methods comprising a first antibody comprising a fully human anti-CD47 antibody and a second antibody that specifically binds a cell surface antigen and comprises an Fc portion that can bind an Fcγ receptor on an effector cell. In various embodiments, the second antibody comprises a tumor-targeting antibody, such as an antibody that binds CD20, PD-L1, CD38 or SLAMF7 antigens.

The fully human anti-CD47 antibody in various embodiments has a heavy chain variable region having at least 95%, at least 96%, at least 97%, at least 98%, or at least 99% identity to SEQ ID NO:1 and a light chain variable region having at least 95%, at least 96%, at least 97%, at least 98%, or at least 99% identity to SEQ ID NO:2. In some embodiments the fully human antibody is an IgG2 antibody or an IgG4 antibody. In some embodiments the fully human antibody is an IgG1 antibody having one or more mutations in the Fc region, where the one or more mutations result in reduced interaction of the Fc region with an Fc receptor.

Also provided herein are methods of treating a subject having cancer, comprising administering a therapeutically effective amount of 1) a first antibody of an antigen binding fragment thereof that binds CD47 and 2) a second antibody that binds an antigen present on a cancer cell, where the first antibody binds to CD47 and blocks binding between CD47 antigen and SIRPα antigen, and the second antibody comprises an Fc region that binds an Fcγ receptor on an effector cell. In various embodiments the first antibody is an anti-CD47 antibody as disclosed herein that comprises a heavy chain variable region having at least 95% identity to SEQ ID NO:1 and a light chain variable region having at least 95% identity to SEQ ID NO:2. In various embodiments the second antibody of the antibody that includes an Fc region binds a tumor antigen, such as CD20, CD38, PD-L1, or SLAMF7. For example, the second antibody can be an anti-CD20 antibody such as rituximab or an anti-CD38 antibody such as Daratumumab.

Also included are methods for killing at least one cancer cell in a population of cancer cells, wherein the at least one cancer cell overexpresses CD47 antigen, the method comprising: contacting the at least one cancer cell with a therapeutically effective amount of a first antibody or an antigen binding fragment thereof that binds CD47 antigen and a second antibody that binds a tumor antigen, where the first antibody binds to CD47 antigen and blocks binding between CD47 antigen and SIRPα antigen, and wherein the second antibody binds a tumor cell and comprises Fc portion that binds an Fcγ receptor on an effector cell.

Also included are methods for treating a subject having a cancer that overexpresses CD47 antigen, the method comprising: administering to the subject a therapeutically effective amount of a first antibody or an antigen binding fragment thereof that binds CD47 antigen and a second antibody that binds a tumor antigen, where the first antibody binds to CD47 antigen and blocks binding between CD47 antigen and SIRPα antigen, and wherein the second antibody binds a tumor cell and comprises Fc portion that binds an Fcγ receptor on an effector cell.

The methods can use any of the CD47 antibodies disclosed herein, such as the STI-6643 antibody and variants thereof, and can use any tumor targeting antibodies, including but not limited to antibodies that specifically bind CD20, CD38, or PD-L1.

### DESCRIPTION OF THE FIGURES

FIG. 1 shows a schematic of a hemagglutination reaction (upper) and a photograph of a hemagglutination assay comparing activity of anti-CD47 antibodies STI-6643 and Hu5F9.

FIG. 2A is a schematic of a competition assay with anti-CD47/SIRP-alpha-Fc for CD47 binding.

FIG. 2B is a graph of the competition assay comparing the activity of anti-CD47 antibodies STI-6643 and Hu5F9.

FIG. 3 is a graph of an antibody dependent cellular phagocytosis (ADCP) assay comparing the activity of anti-CD47 antibodies STI-6643 and Hu5F9.

FIG. 4 is a bar graph comparing increases in phagocytosis killing in assays testing the combination of anti-CD47 antibody clone STI-6643 and suboptimal amounts of anti-CD20 antibody Rituximab.

FIG. 5A shows anti-tumor activity in a disseminated human Raji-Fluc xenograft mouse model comparing the activity of a control isotype IgG4, anti-CD47 antibodies STI-6643 and Hu5F9.

FIG. 5B are graphs showing the anti-tumor activity of the mouse model described in FIG. 5A. The upper graph shows total flux detected in mice treated with a control isotype IgG4 or anti-CD47 antibody clone STI-6643. The lower graph shows the total flux detected in mice treated with a control isotype IgG4 or anti-CD47 antibody Hu5F9. See Example 5.

FIG. 5C is a graph showing a statistical significance analysis of the data shown in FIGS. 5B and 5C.

FIG. 5D is a graph showing animal survival analysis based on the data shown in FIGS. 5A-C.

FIG. 5E is a graph of circulating antibody detection in the animals described in FIGS. 5A-C.

FIG. 6A shows anti-tumor activity in a disseminated human Raji-Fluc xenograft mouse model comparing the activity of a control isotype IgG4, anti-CD47 antibodies STI-6643 or Hu5F9 as mono-therapy, or the combination of anti-CD47 antibodies STI-6643 and Hu5F9. See Example 6.

FIG. 6B are graphs showing the anti-tumor activity of the mouse model described in FIG. 6A. The left graph shows total flux detected in mice treated with a control isotype IgG4 or anti-CD47 antibody clone STI-6643 as a mono-therapy. The middle graph shows total flux detected in mice treated with a control isotype IgG1 or anti-CD20 antibody Rituximab as a mono-therapy. The right graph shows the total flux detected in mice treated with a combination control isotype IgG1 and IgG4 isotype, or the combination of anti-CD47 antibody clone STI-6643 and anti-CD20 antibody Rituximab.

FIG. 6C is a graph showing a statistical significance analysis of the data shown in FIGS. 6B and C.

FIG. 6D is a graph showing animal survival analysis based on the data shown in FIGS. 6A-C.

FIG. 7A is a graph reproduced from Liu, et al., 2015 PLoS ONE (10)9: e0137345 (doi:10.1371/journal.pone.0137345 (see FIG. 4A in Liu which shows pharmacokinetic analysis (hemoglobin) of cynomolgus monkeys administered single intravenous infusions of anti-CD47 antibody Hu5F9 at doses indicated in the figure. The shaded bar indicates the range of hemoglobin that might trigger transfusion in humans).

FIG. 7B is a graph showing our pharmacokinetic analysis of cynomolgus monkeys administered anti-CD47 antibody STI-6643 (each dose at 150 mg/kg) once weekly via IV bolus for four weeks. The shaded bar indicates the range of hemoglobin that might trigger transfusion in humans).

FIG. 8A contains four graphs showing preferential binding of anti-CD47 antibody clone STI-6643 to tumor cells with respect to red blood cells (RBCs) as compared to anti-CD47 antibody Hu5F9 binding to tumor cells and RBCs. The graphs display the results of flow cytometry data from a binding assay on mixed-cell samples.

FIG. 8B is a bar graph showing binding of anti-CD47 antibody clone STI-6643 to RBCs and tumor cells (Raji, CD19-expressing tumor cells, and CD3-expressing tumor cells) by anti-CD47 antibody clone STI-6643. The binding of antibody Hu5F9 to Raji cells is set at 100% on the y- axis for comparison. FIG. 9 shows a schematic of a hemagglutination reaction (upper) and a photograph of another hemagglutination assay comparing activity of anti-CD47 antibodies STI-6643 and Hu5F9.

FIG. 10 shows four graphs from a three-way mixed lymphocyte reaction (MLR) assay.

FIG. 11A is a bar graph showing the results of a Staphylococcal Enterotoxin B (SEB) assay. Each concentration along the x-axis includes from left to right: no antibody control; isotype IgG4 control; Hu5F9; and STI-6643.

FIG. 11B shows the results of the SEB assay described in FIG. 11A above, with the number of CD4+ and CD8+ T cells shown in two separate graphs.

FIG. 11C shows the results of the SEB assay described in FIG. 11A above, with the number of CD25+ CD4+ and CD25+ CD8+ activated T cells shown in two separate graphs.

FIG. 12A is a graph showing the percent survival from a dose study in a Raji mouse tumor model.

FIG. 12B is a Table listing the p values of each treatment group in the mouse Raji tumor model described in FIG. 12A above.

FIG. 12C is a graph showing the cumulative circulating concentration of antibody from the Raji mouse tumor model described in FIG. 12A above.

FIG. 13A is a graph showing the average tumor volume from an efficacy study in mouse NCI-H82 lung solid tumor model.

FIG. 13B shows tumor volumes from individual animals treated with either isotype IgG4 antibody or STI-6643 antibody, in the mouse NCI-H82 lung solid tumor model described in FIG. 13A above.

FIG. 13C is a bar graph showing the relative tumor weight from the mouse NCI-H82 lung solid tumor model described in FIG. 13A above.

FIG. 13D is a bar graph showing the circulating antibody concentrations from the mouse NCI-H82 lung solid tumor model described in FIG. 13A above. Each time post along the x-axis includes from left to right: isotype IgG4 control; and STI-6643.

FIG. 14 shows several graphs of tumor volume and percent survival from a dose efficacy study in a mouse NCI-H82 lung solid tumor model.

FIG. 15A is a graph showing tumor volume from an efficacy study in a mouse A375 melanoma solid tumor study.

FIG. 15B shows tumor volumes from individual animals treated with either isotype IgG4 antibody or STI-6643 antibody, in the mouse A375 melanoma solid tumor study described in FIG. 15A above.

FIG. 15C shows a percent survival graph from the mouse A375 melanoma solid tumor study described in FIG. 15A above.

FIG. 16A shows a percent survival graphs from an efficacy study in a mouse Raji tumor model in which the mice were treated with a combination of STI-6643 and an anti-CD38 antibody (Daratumumab).

FIG. 16B is a Table listing the p values of each treatment group in the mouse combination therapy study described in FIG. 16A above.

FIG. 17A provides examples of positive and negative hemagglutination assays.

FIG. 17B provides a picture of the results of hemagglutination assays using anti-CD47 antibodies STI-6643, Hu5F9, AO-176, and 13H3.

FIG. 17C provides pictures of the results of hemagglutination assays using anti-CD47 antibodies STI-6643 and Hu5F9 with human, cynomolgus, and canine RBCs.

FIG. 18 provides graphs of binding of anti-CD47 antibodies STI-6643 and Hu5F9 to human, cynomolgus, and canine RBCs as a function of antibody concentration.

FIG. 19 provides graphs of binding of anti-CD47 antibodies STI-6643, Hu5F9, AO-176, and 13H3 to Raji tumor cells and RBCs as a function of antibody concentration.

FIG. 20A provides graphs of numbers of CD4+, CD8+, CD19+, and CD56+ cells recovered from PBMCs after incubation with anti-CD47 antibodies STI-6643, Hu5F9, AO-176, and 13H3.

FIG. 20B provides graphs of CD4+, CD8+, CD19+, and CD56+ cells recovered from PBMCs after incubation with anti-CD47 antibodies STI-6643, Hu5F9, AO-176, and 13H3 as a percentage of the same cell types recovered after incubation with the isotype control.

FIG. 21 provides graphs of tumor volume over time in tumor-bearing mice treated with different dosages of anti-CD47 antibodies.

FIGS. 22A-C show the amino acid sequences of various anti-CD47 antibodies, a CD47 antigen, anti-CD20 antibodies and a CD20 antigen.

FIGS. 23A-E show the amino acid sequences of various anti-CD38 antibodies and CD38 target antigens.

### DESCRIPTION

Headings provided herein are solely for the convenience of the reader and do not limit the various aspects of the disclosure, which aspects can be understood by reference to the specification as a whole.

The disclosures of all publications, patents, and patent applications cited herein are hereby incorporated by reference in their entireties into this application.

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) -Antibody Engineering, 2nd Edition Freeman and Company, NY, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif, and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation and delivery, and treatment of patients.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

The terms “peptide”, “polypeptide” and “protein” and other related terms used herein are used interchangeably and refer to a polymer of amino acids that is not limited to any particular length. Polypeptides may comprise natural and non-natural amino acids. Polypeptides include recombinant and chemically-synthesized polypeptides. Polypeptides include precursor molecules and mature (e.g., processed) molecules. Precursor molecules include those that have not yet been subjected to cleavage, for example cleavage of a secretory signal peptide or by enzymatic or non-enzymatic cleavage at certain amino acid residue(s). Polypeptides include mature molecules that have undergone cleavage. These terms encompass native proteins, recombinant proteins, and artificial proteins, protein fragments and polypeptide analogs (such as muteins, variants, chimeric proteins and fusion proteins) of a protein sequence as well as post-translationally, or otherwise covalently or non-covalently, modified proteins.

The terms “nucleic acid”, “nucleic acid molecule”, “polynucleotide” and “oligonucleotide” and other related terms used herein are used interchangeably and refer to polymers of nucleotides that are not limited to any particular length. Nucleic acids include recombinant and chemically-synthesized forms. Nucleic acids include DNA molecules (e.g., cDNA or genomic DNA, expression constructs, DNA fragments, etc.), RNA molecules (e.g., mRNA), analogs of the DNA or RNA generated using nucleotide analogs (e.g., peptide nucleic acids and non-naturally occurring nucleotide analogs), and hybrids thereof, as well as peptide nucleic acids, locked nucleic acids, and other synthetic nucleic acid analogs and hybrids thereof. A nucleic acid molecule can be single-stranded or double-stranded. In one embodiment, the nucleic acid molecules of the disclosure comprise a contiguous open reading frame encoding an antibody, or a fragment or scFv, derivative, mutein, or variant thereof. In some embodiments, nucleic acids comprise one type of polynucleotides or a mixture of two or more different types of polynucleotides.

The term “recover” or “recovery” or “recovering”, and other related terms, refer to obtaining a protein (e.g., an antibody or an antigen binding portion thereof), from host cell culture medium or from host cell lysate or from the host cell membrane. In one embodiment, the protein is expressed by the host cell as a recombinant protein fused to a secretion signal peptide sequence (e.g., leader peptide sequence) which mediates secretion of the expressed protein. The secreted protein can be recovered from the host cell medium. In one embodiment, the protein is expressed by the host cell as a recombinant protein that lacks a secretion signal peptide sequence which can be recovered from the host cell lysate. In one embodiment, the protein is expressed by the host cell as a membrane-bound protein which can be recovered using a detergent to release the expressed protein from the host cell membrane. In one embodiment, irrespective of the method used to recover the protein, the protein can be subjected to procedures that remove cellular debris from the recovered protein. For example, the recovered protein can be subjected to chromatography, gel electrophoresis and/or dialysis. In one embodiment, the chromatography comprises any one or any combination or two or more procedures including affinity chromatography, hydroxyapatite chromatography, ion-exchange chromatography, reverse phase chromatography and/or chromatography on silica. In one embodiment, affinity chromatography comprises protein A or protein G (cell wall components from Staphylococcus aureus).

The term “isolated” refers to a protein (e.g., an antibody or an antigen binding portion thereof) or polynucleotide that is substantially free of other cellular material. The term isolated also refers in some embodiments to protein or polynucleotides that are substantially free of other molecules of the same species, for example other proteins or polynucleotides having different amino acid or nucleotide sequences, respectively. The purity or homogeneity of the desired molecule can be assayed using techniques well known in the art, including low resolution methods such as gel electrophoresis and high resolution methods such as HPLC or mass spectrometry. In various embodiments any of the anti-CD47 antibodies or tumor targeting antibodies disclosed herein are isolated.

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

The term “leader sequence” or “leader peptide” or “[peptide] signal sequence” or “signal peptide” or “secretion signal peptide” refers to a peptide sequence that is located at the N-terminus of a polypeptide. A leader sequence directs a polypeptide chain to a cellular secretory pathway and can direct integration and anchoring of the polypeptide into the lipid bilayer of the cellular membrane. Typically, a leader sequence is about 10-50 amino acids in length and is cleaved from the polypeptide upon secretion of the mature polypeptide or insertion of the mature polypeptide into the membrane. Thus, proteins provided herein such as membrane proteins and antibodies having signal peptides that are identified by their precursor sequences that include a signal peptide sequence are also intended to encompass the mature forms of the polypeptides lacking the signal peptide, and proteins provided herein such as membrane proteins and antibodies having signal peptides that are identified by their mature polypeptide sequences that lack a signal peptide sequence are also intended to encompass forms of the polypeptides that include a signal peptide, whether native to the protein or derived from another secreted or membrane-inserted protein.. In one embodiment, a leader sequence includes signal sequences comprising CD8a, CD28 or CD16 leader sequences. In one embodiment, the signal sequence comprises a mammalian sequence, including for example mouse or human Ig gamma secretion signal peptide. In one embodiment, a leader sequence comprises a mouse Ig gamma leader peptide sequence

### MEWS WVFLFFLSVTTGVHS (SEQ ID NO:40).

An “antigen-binding protein” and related terms used herein refer to a protein comprising a portion that binds to an antigen and, optionally, a scaffold or framework portion that allows the antigen binding portion to adopt a conformation that promotes binding of the antigen-binding protein to the antigen. Examples of antigen-binding proteins include antibodies, antibody fragments (e.g., an antigen binding portion of an antibody), antibody derivatives, and antibody analogs. As used herein an “antigen-binding protein derived from [a referenced] antibody” is an antigen-binding protein that includes the variable light chain sequence and variable heavy chain sequence of the referenced antibody. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold.

An antigen binding protein can have, in some examples, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The heavy and/or light chains may or may not include a leader sequence for secretion. The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules.

The variable regions of immunoglobulin chains exhibit the same general structure of three hypervariable regions, also called complementarity determining regions or CDRs, joined by relatively conserved framework regions (FR). From N-terminus to C-terminus, both light and heavy chains comprise the segments FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991 (e.g., “Kabat numbering”). Other numbering systems for the amino acids in immunoglobulin chains include IMGT.RTM. (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 19971 Mol. Biol. 273:927-948; Contact (Maccallum et al., 19961 Mol. Biol. 262:732-745, and Aho (Honegger and Pluckthun 2001 J. Mol. Biol. 309:657-670.

An “antibody” and “antibodies” and related terms used herein refers to an intact immunoglobulin or to an antigen binding portion thereof (or an antigen binding fragment thereof) that binds specifically to an antigen. Antigen binding portions (or the antigen binding fragment) may be produced by recombinant DNA techniques or by enzymatic or chemical cleavage of intact antibodies. Antigen binding portions (or antigen binding fragments) include, inter alia, Fab, Fab′, F(ab′)₂, Fv, single domain antibodies (dAbs), and complementarity determining region (CDR) fragments, single-chain antibodies (scFv), chimeric antibodies, diabodies, triabodies, tetrabodies, nanobodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)2 fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal antibody populations.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Monoclonal antibodies include monoclonal antibodies produced using hybridoma methods that provide a cell line producing a population of identical antibody molecules, and also include chimeric, hybrid, and recombinant antibodies produced by cloning methods such that a cell transfected with the construct or constructs that include the antibody-encoding sequences and the progeny of the transfected cell produce a population of antibody molecules directed against a single antigenic site. For example, variable regions of an antibody (variable heavy chain and light chain regions or variable heavy and light chain CDRs) may be cloned into an antibody framework that includes constant regions of any species, including human constant regions, where expression of the construct in a cell can produce a single antibody molecule or antigen-binding protein that is referred to herein as monoclonal.

The modifier “monoclonal” thus indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler and Milstein, Nature, 256:495 (1975), or may be made by recombinant DNA methods such as described in U.S. Pat. No. 4,816,567. The “monoclonal antibodies” may also be isolated from phage libraries generated using the techniques described in McCafferty et al., Nature, 348:552-554 (1990), for example.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In various embodiments, an antibody specifically binds to a target antigen if it binds to the antigen with a dissociation constant (K) of 10⁻⁵ M or less, or 10⁻⁶ M or less, or 10⁻⁷ M or less, or 10⁻⁸ M or less, or 10⁻⁹ M or less, or 10⁻¹⁰ M or less, or 10⁻¹¹ or less, or 10⁻¹² or less.

Binding affinity of an antigen-binding protein for a target antigen can be reported as a dissociation constant (K_{d}) which can be measured using a surface plasmon resonance (SPR) assay. Surface plasmon resonance refers to an optical phenomenon that allows for the analysis of real-time interactions by detection of alterations in protein concentrations within a biosensor matrix, for example using a BIACORE system (Biacore Life Sciences division of GE Healthcare, Piscataway, N.J.).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope.

With respect to antibodies, the term “antagonist” and “antagonistic” refers to a blocking antibody that binds its cognate target antigen and inhibits or reduces the biological activity of the bound antigen. The term “agonist” or “agonistic” refers to an antibody that binds its cognate target antigen in a manner that mimics the binding of the physiological ligand which causes antibody-mediated downstream signaling.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a VL domain, or an antigen-binding fragment of a VH or VL domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989).

A single-chain antibody (scFv) is an antibody in which a V_{L} and a V_{H} region are joined via a linker (e.g., a synthetic sequence of amino acid residues) to form a continuous protein chain. In one embodiment, the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83).

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different. Diabody, tribody and tetrabody constructs can be prepared using antigen binding portions from any of the anti-CD47 antibodies described herein.

A “humanized antibody” refers to an antibody originating from a non-human species that has one or more variable and constant regions that has been sequence modified to conform to corresponding human immunoglobulin amino acid sequences. For example, the constant regions of a humanized antibody may be human constant region sequences, where the amino acid sequence of a variable domains may be from an antibody sequence of another species, such as a mouse (in which the antibody may have been generated). A humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In some embodiments, the constant domain(s) from a human antibody are fused to the variable domain(s) of a non-human species. In some embodiments, one or more amino acid residues in one or more CDR sequences of a non-human antibody is changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

In some embodiments, an antibody can be a “fully human” antibody in which all of the constant and variable domains (optionally excepting from the CDRs) are derived from human immunoglobulin sequences. A fully human antibody as disclosed herein may have one or more mutations (which may be, for example amino acid substitutions, deletions, or insertions) in the constant regions, such as for example the Fc constant regions of the heavy chain, with respect to a wild type human antibody sequence. For example, a fully human antibody can have one or more mutation in the constant regions of either the light or heavy chain of the antibody, where the sequence of either or both of the light chain constant region or heavy chain constant regions (CH1, CH2, and CH3) of the fully human antibody are greater than 95%, greater than 96%, greater than 97%, and preferably greater than 98% or at least 99% identical to the sequence of the non-mutant human constant regions. Humanized and fully human antibodies may be prepared in a variety of ways, examples of which are described below, including through recombinant methodologies or through immunization with an antigen of interest of a mouse that is genetically modified to express antibodies derived from human heavy and/or light chain-encoding genes, e.g., the “Xenomouse II” that, when challenged with an antigen, generates high affinity fully human antibodies Mendez et al. ((1997) Nature Genetics 15: 146-156). This was achieved by germ-line integration of megabase human heavy chain and light chain loci into mice with deletion of the endogenous J_{H} region. The antibodies produced in these mice closely resemble that seen in humans in all respects, including gene rearrangement, assembly, and repertoire.

Alternatively, phage display technology (McCafferty et al., Nature 348, 552-553 [1990]) can be used to produce human antibodies and antibody fragments in vitro, from immunoglobulin variable (V) domain gene repertoires from immunized or nonimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; see, e.g., Johnson, Kevin S. and Chiswell, David J., Current Opinion in Structural Biology 3, 564-571 (1993). Any of a number of sources of V-gene segments can be used for phage display, e.g., the spleens of immunized mice (Clackson et al., Nature 352, 624-628 (1991)) or blood cells of nonimmunized human donors can be used to generate antibodies to a diverse array of antigens (including self-antigens) can be isolated essentially following the techniques described by Marks et al., J. Mol. Biol. 222, 581-597 (1991) or Griffith et al., EMBO J. 12, 725-734 (1993).

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions of a chimeric antibody may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen).

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation.

Unless otherwise indicated, the term “antibody” includes, in addition to antibodies comprising full-length heavy chains and full-length light chains, derivatives, variants, fragments, and muteins thereof, examples of which are described below.

The term “hinge” refers to an amino acid segment that is generally found between two domains of a protein and may allow for flexibility of the overall construct and movement of one or both of the domains relative to one another. Structurally, a hinge region comprises from about 10 to about 100 amino acids, e.g., from about 15 to about 75 amino acids, from about 20 to about 50 amino acids, or from about 30 to about 60 amino acids. In one embodiment, the hinge region is 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids in length. The hinge region can be derived from is a hinge region of a naturally-occurring protein, such as a CD8 hinge region or a fragment thereof, a CD8a hinge region, or a fragment thereof, a hinge region of an antibody (e.g., IgG, IgA, IgM, IgE, or IgD antibodies), or a hinge region that joins the constant domains CH1 and CH2 of an antibody. The hinge region can be derived from an antibody and may or may not comprise one or more constant regions of the antibody, or the hinge region comprises the hinge region of an antibody and the CH3 constant region of the antibody, or the hinge region comprises the hinge region of an antibody and the CH2 and CH3 constant regions of the antibody, or the hinge region is a non-naturally occurring peptide, or the hinge region is disposed between the C-terminus of the scFv and the N-terminus of the transmembrane domain. In one embodiment, the hinge region comprises any one or any combination of two or more regions comprising an upper, core or lower hinge sequences from an IgG1, IgG2, IgG3 or IgG4 immunoglobulin molecule. In one embodiment, the hinge region comprises an IgG1 upper hinge sequence EPKSCDKTHT (SEQ ID NO:41). In one embodiment, the hinge region comprises an IgG1 core hinge sequence CPXC, wherein X is P, R or S. In one embodiment, the hinge region comprises a lower hinge/CH2 sequence PAPELLGGP ((SEQ ID NO:42)). In one embodiment, the hinge is joined to an Fc region (CH2) having the amino acid sequence SVFLFPPKPKDT (SEQ ID NO:43). In one embodiment, the hinge region includes the amino acid sequence of an upper, core and lower hinge and comprises EPKSCDKTHTCPPCPAP ELLGGP (SEQ ID NO:44). In one embodiment, the hinge region comprises one, two, three or more cysteines that can form at least one, two, three or more interchain disulfide bonds.

The term “Fc” or “Fc region” as used herein refers to the portion of an antibody heavy chain constant region beginning in or after the hinge region and ending at the C-terminus of the heavy chain. The Fc region comprises at least a portion of the CH2 and CH3 regions and may, or may not, include a portion of the hinge region. An Fc domain can bind Fc cell surface receptors and some proteins of the immune complement system. An Fc region can bind a complement component C1q. An Fc domain exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. An Fc domain can bind an Fc receptor, including FcγRI (e.g., CD64), FcγRII (e.g, CD32) and/or FcγRIII (e.g., CD16a). An Fc region can include a mutation that increases or decreases any one or any combination of these functions. For example, the Fc region can comprise a LALA mutation (e.g., equivalent to L234A, L235A according to Kabat numbering) which reduces effector function. In one example, the Fc domain comprises a LALA-PG mutation (e.g., equivalent to L234A, L235A, P329G according to Kabat numbering) which reduces effector function. An Fc domain can also include one or more mutations that can increase or decrease the serum half-life of the antibody.

The term “labeled” or related terms as used herein with respect to a polypeptide refers to joinder antibodies and their antigen binding portions thereof that are unlabeled or joined to a detectable label or moiety for detection, wherein the detectable label or moiety is radioactive, colorimetric, antigenic, enzymatic, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the anti-PD-1 antibodies described herein can be unlabeled or can be joined to a detectable label or moiety.

The term “labeled” or related terms as used herein with respect to a polypeptide refers to joinder thereof to a detectable label or moiety for detection. Exemplary detectable labels or moieties include radioactive, colorimetric, antigenic, enzymatic labels/moieties, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the anti-CD47 antibodies described herein or tumor antigen-binding antibodies that are described herein can be unlabeled or can be joined to a detectable label or detectable moiety.

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters. Expressions such as “comprises a sequence with at least X% identity to Y” with respect to a test sequence mean that, when aligned to sequence Y as described above, the test sequence comprises residues identical to at least X% of the residues of Y.

In one embodiment, the amino acid sequence of a test antibody may be similar but not necessarily identical to any of the amino acid sequences of the polypeptides that make up any of the anti-CD47 antibodies described herein. The similarities between the test antibody and the polypeptides can be at least 95%, or at or at least 96% identical, or at least 97% identical, or at least 98% identical, or at least 99% identical, to any of the polypeptides that make up any of the anti-CD47 antibodies, or antigen binding protein thereof, described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

A “vector” and related terms used herein refers to a nucleic acid molecule (e.g., DNA or RNA) which can be operably linked to foreign genetic material (e.g., nucleic acid transgene). Vectors can be used as a vehicle to introduce foreign genetic material into a cell (e.g., host cell). Vectors can include at least one restriction endonuclease recognition sequence for insertion of the transgene into the vector. Vectors can include at least one gene sequence that confers antibiotic resistance or a selectable characteristic to aid in selection of host cells that harbor a vector-transgene construct. Expression vectors can include one or more origin of replication sequences. Vectors can be single-stranded or double-stranded nucleic acid molecules. Vectors can be linear or circular nucleic acid molecules. One type of vector is a “plasmid,” which refers to a linear or circular double stranded extrachromosomal DNA molecule which can be linked to a transgene, and is capable of replicating in a host cell, and transcribing and/or translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated viral, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can contain one or more regulatory sequences, such as inducible and/or constitutive promoters and enhancers. Expression vectors can include ribosomal binding sites and/or polyadenylation sites. Expression vectors can include one or more origin of replication sequences. Regulatory sequences direct transcription, or transcription and translation, of a transgene linked to or inserted into the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif and Baron et al., 1995, Nucleic Acids Res. 23:3605-3606.

A transgene is “operably linked” to a regulatory sequence (e.g., a promoter) when the regulatory sequence affects the expression (e.g., the level, timing, or location of expression) of the transgene.

The terms “transfected” or “transformed” or “transduced” or other related terms used herein refer to a process by which exogenous nucleic acid (e.g., transgene) is transferred or introduced into a host cell, such as an antibody production host cell. A “transfected” or “transformed” or “transduced” host cell is one which has been introduced with exogenous nucleic acid (transgene). The host cell includes the primary subject cell and its progeny. Exogenous nucleic acids encoding at least a portion of any of the anti-CD47 antibodies described herein can be introduced into a host cell. Expression vectors comprising at least a portion of any of the anti-CD47 antibodies described herein can be introduced into a host cell, and the host cell can express polypeptides comprising at least a portion of the anti-CD47 antibody.

In this context, a host cell can be a cultured cell that can be transformed or transfected with a polypeptide-encoding nucleic acid, which can then be expressed in the host cell. The phrase “transgenic host cell” or “recombinant host cell” can be used to denote a host cell that has been introduced (e.g., transduced, transformed or transfected) with a nucleic acid either to be expressed or not to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

Thus the terms “host cell” or “or a population of host cells” or related terms as used herein may refer to a cell (or a population thereof or a plurality of host cells) to be used for production of the antibody or fragment thereof, is a cell or cells into which foreign (exogenous or transgene) nucleic acids have been introduced, for example, to direct production of the anti-CD47 antibody by the production host cell. The foreign nucleic acids can include an expression vector operably linked to a transgene, and the host cell can be used to express the nucleic acid and/or polypeptide encoded by the foreign nucleic acid (transgene). A host cell (or a population thereof) can be a cultured cell, can be extracted from a subject, or can be the cell of an organism, including a human subject. The host cell (or a population of host cells) includes the primary subject cell and its progeny without any regard for the number of generations or passages. The host cell (or a population thereof) includes immortalized cell lines. Progeny cells may or may not harbor identical genetic material compared to the parent cell. In one embodiment, a production host cell describes any cell (including its progeny) that has been modified, transfected, transduced, transformed, and/or manipulated in any way to express an antibody, as disclosed herein. In one example, the host cell (or population thereof) can be transfected or transduced with an expression vector operably linked to a nucleic acid encoding the desired antibody, or an antigen binding portion thereof, as described herein. Production host cells and populations thereof can harbor an expression vector that is stably integrated into the host's genome or can harbor an extrachromosomal expression vector. In one embodiment, host cells and populations thereof can harbor an extrachromosomal vector that is present after several cell divisions or is present transiently and is lost after several cell divisions.

The term “subject” as used herein refers to human and non-human animals, including vertebrates, mammals, and non-mammals. In one embodiment, the subject can be human, non-human primates, simian, ape, murine (e.g., mice), bovine, porcine, equine, canine, feline, caprine, lupine, ranine, or piscine.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Any of the anti-CD47 antibodies described herein (or tumor antigen binding antibodies disclosed herein) can be administered to a subject using art-known methods and delivery routes.

The terms “effective amount”, “therapeutically effective amount” or “effective dose” or related terms may be used interchangeably and refer to an amount of antibody or an antigen binding protein (e.g., any of the anti-CD47 antibodies described herein or tumor antigen-binding antibodies disclosed herein) that when administered to a subject, is sufficient to effect a measurable improvement or prevention of a disease or disorder associated with tumor or cancer antigen expression. Therapeutically effective amounts of antibodies provided herein, when used alone or in combination, will vary depending upon the relative activity of the antibodies and combinations (e.g. , in inhibiting cell growth) and depending upon the subject and disease condition being treated, the weight and age and sex of the subject, the severity of the disease condition in the subject, the manner of administration and the like, which can readily be determined by one of ordinary skill in the art.

In one embodiment, a therapeutically effective amount will depend on certain aspects of the subject to be treated and the disorder to be treated and may be ascertained by one skilled in the art using known techniques. In general, the polypeptide is administered to a subject at about 0.01 g/kg-50 mg/kg per day, about 0.01 mg/kg-30 mg/kg per day, or about 0.1 mg/kg-20 mg/kg per day. The polypeptide may be administered daily (e.g., once, twice, three times, or four times daily) or less frequently (e.g., weekly, every two weeks, every three weeks, monthly, or quarterly). In addition, as is known in the art, adjustments for age as well as the body weight, general health, sex, diet, time of administration, drug interaction, and the severity of the disease may be necessary.

### Antibody Combination

Provided herein is a composition comprising at least two antibodies, where one antibody is an anti-CD47 antibody that blocks binding of CD47 to the Fcγ receptor (e.g., an FcγRI, FcγRII, or FcγRIII) and a second antibody of the composition specifically binds a tumor antigen and includes an Fc region.

The anti-CD47 antibody can be any described herein, such as an antibody having a heavy chain variable region having at least 95%, at least 96%, at least 97%, at least 98%, or at least 99% identity to SEQ ID NO:1 and a light chain variable region having at least 95%, at least 96%, at least 97%, at least 98%, or at least 99% identity to SEQ ID NO:2. In some embodiments the anti-CD47 antibody is the C47A8-CL antibody (U.S. Pat. No. 10,035,855, incorporated herein by reference) having a heavy chain variable region sequence of SEQ ID NO:1 and a light chain variable region sequence of SEQ ID NO:2 or a variant thereof having a heavy chain variable region having at least 98% or at least 99% identity to SEQ ID NO:1 and a light chain variable region having at least 98% or at least 99% identity to SEQ ID NO:2. The anti-CD47 antibody can be an IgG2 or IgG4 antibody, for example may be an IgG4 antibody.

In some embodiments the antigen-binding protein is an IgG1 antibody having one or more mutations in the Fc region, for example one or more mutations that decreases interaction with an Fcγ receptor and/or one or more mutations that increases antibody half-life. Mutations that reduce or eliminate interaction of the Fc region of an antibody with its receptor (e.g., FcγRs) include, without limitation L234A; L235A or L235E; N297A, N297Q, or N297D; and P329A or P329G. For example, the anti-CD47 antibody can include the mutations L234A and L235A (LALA).

In some alternative embodiments the anti-CD47 antibody can be a single chain antibody, e.g., an ScFv having a heavy chain variable region sequence having at least 95%, at least 96%, at least 97%, at least 98%, or at least 99% identity to SEQ ID NO:1 and a light chain variable region sequence having at least 95%, at least 96%, at least 97%, at least 98%, or at least 99% identity to SEQ ID NO:2. In further embodiments the anti-CD47 antibody can be a Fab fragment of an antibody, e.g., of an IgG antibody having a heavy chain variable region having at least 95%, at least 96%, at least 97%, at least 98%, or at least 99% identity to SEQ ID NO:1 and a light chain variable region having at least 95%, at least 96%, at least 97%, at least 98%, or at least 99% identity to SEQ ID NO:2.

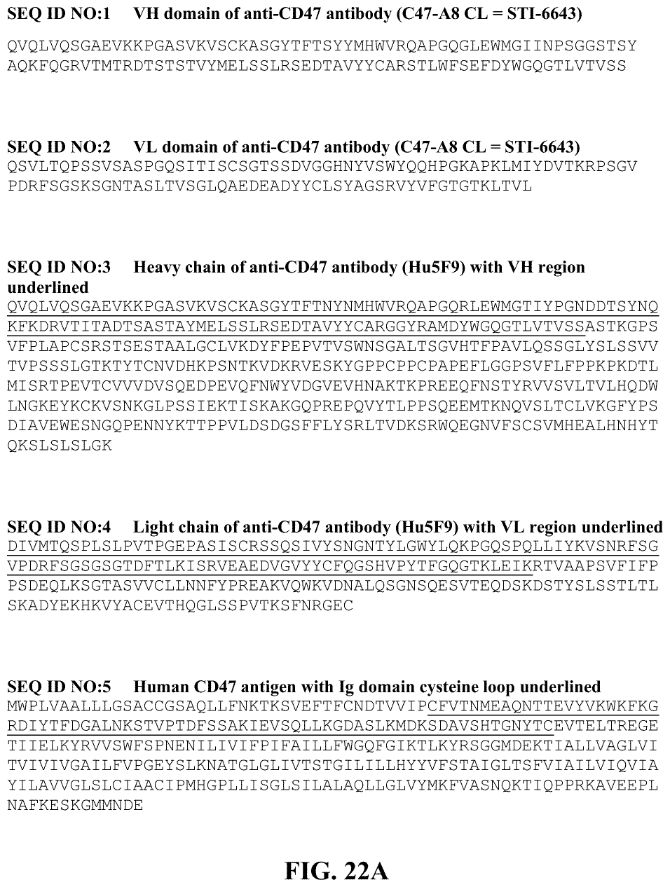

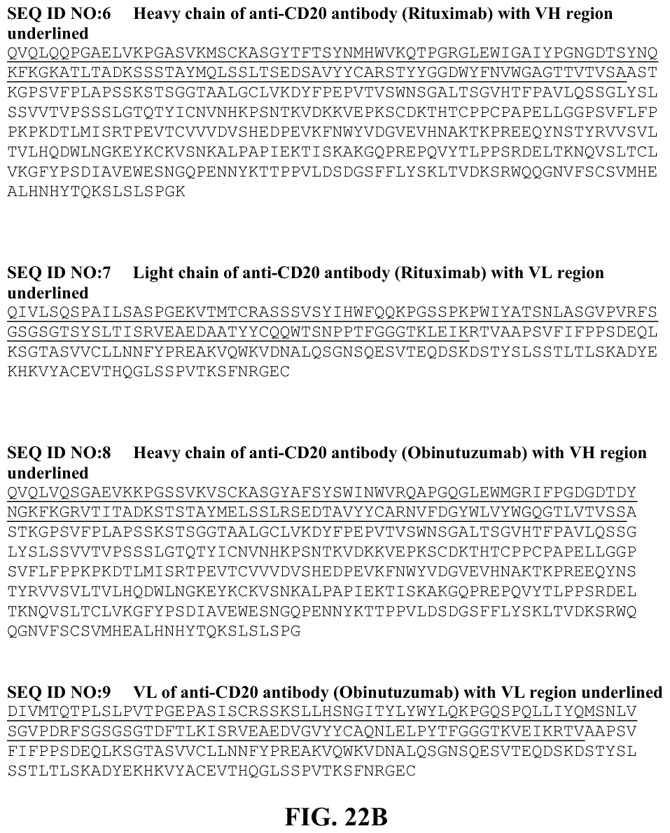

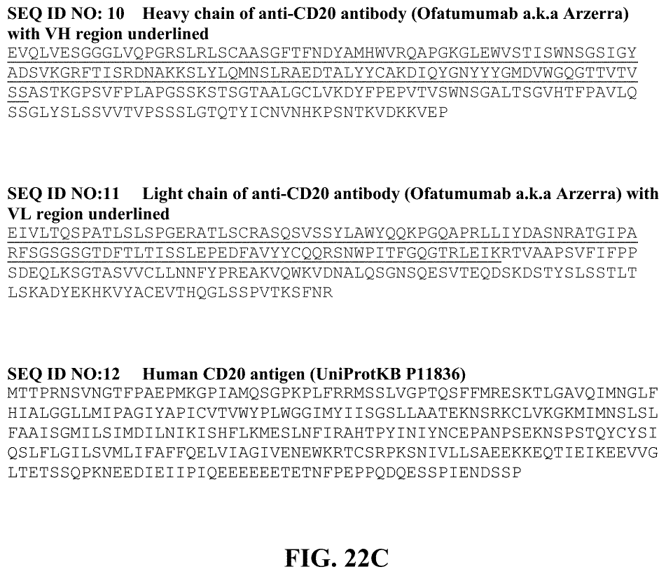

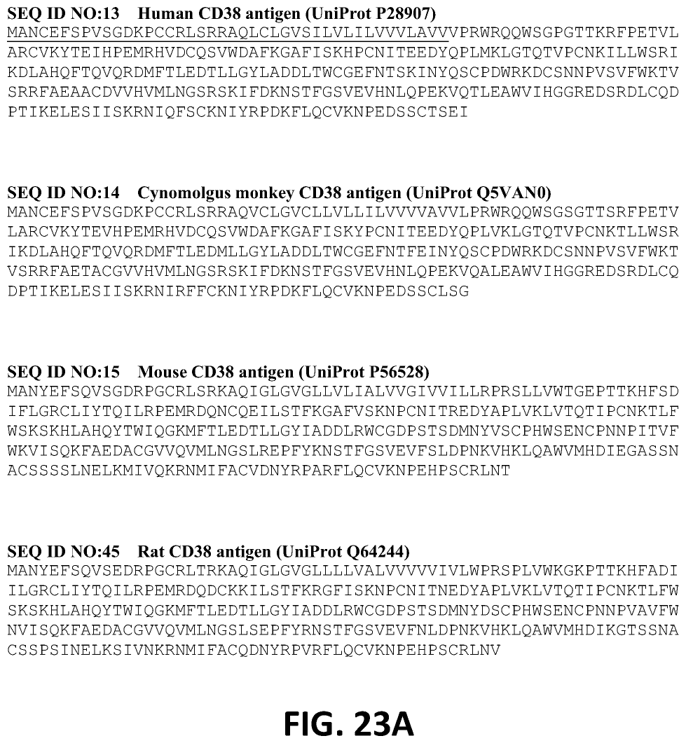

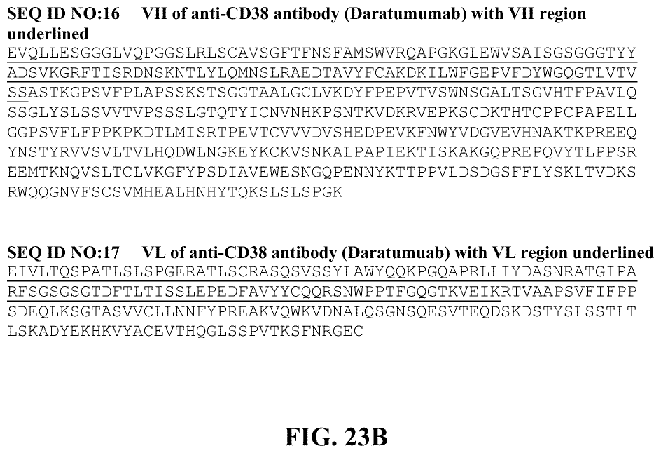

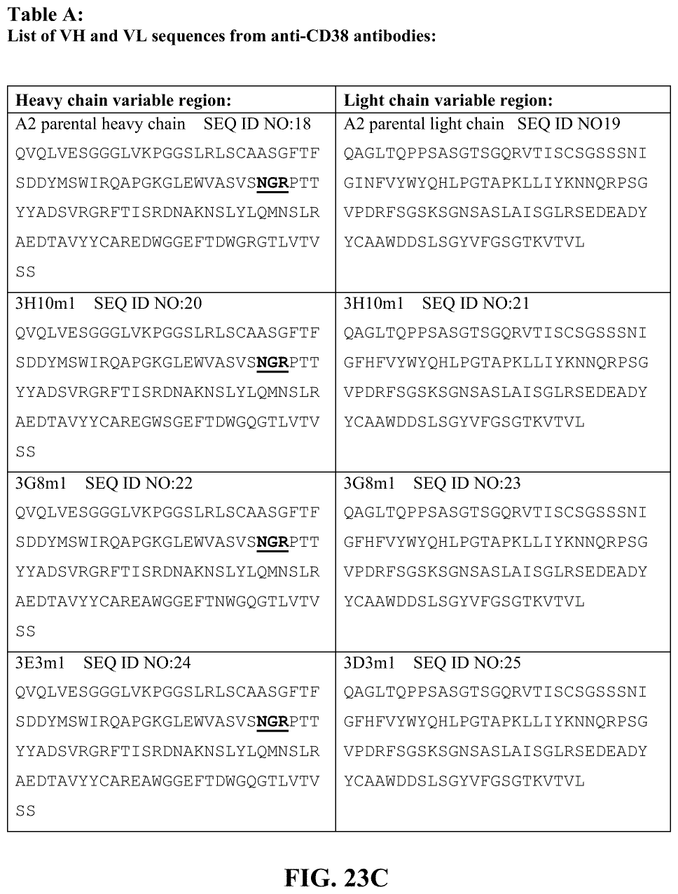

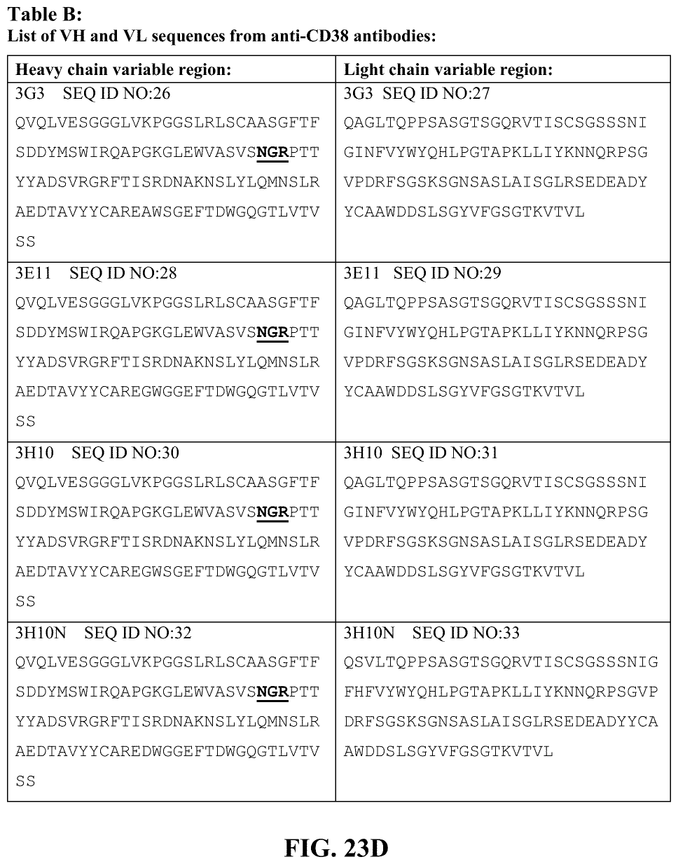

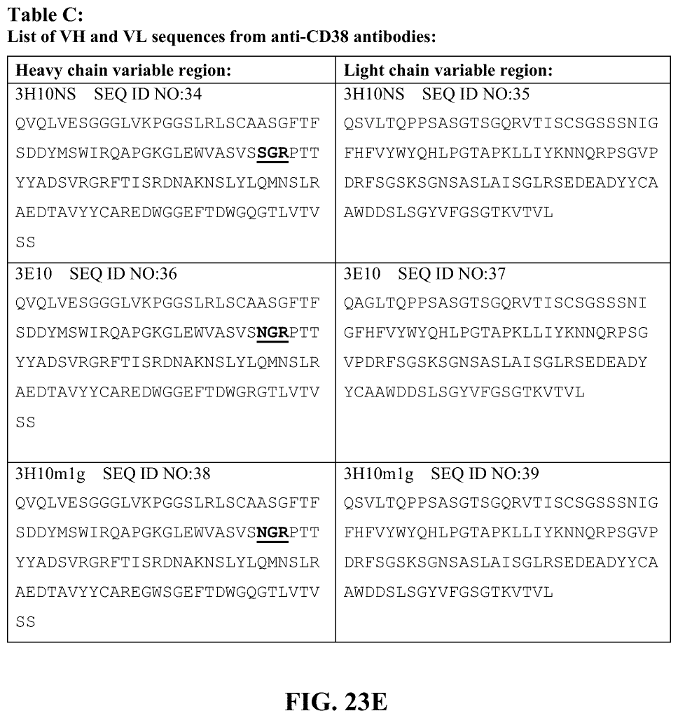

The antibody that binds a tumor antigen and includes an Fc region that binds an Fcγ receptor can be, for example, and IgG1 antibody, and can optionally be an opsonizing antibody that marks the cell to which it binds for destruction by the immune system by means of antibody-dependent cellular cytotoxicity (ADCC) or other mechanisms. The tumor antigen-binding antibody can specifically bind a cell surface antigen expressed on a solid or liquid tumor. For example, the antibody can be an antibody that specifically binds CD19, CD20, CD33, CD38, PD-L1, or SLAMF7. An anti-CD20 antibody can be, as nonlimiting examples, rituximab, ocrelizumab, obinutuzumab, ofatumumab, ibritumomab tiuxetan, tositumomab, or ublituximab, or any of the anti-CD20 antibodies having heavy and light chain variable sequences disclosed in FIG. 22. An anti-CD38 antibody can be, as nonlimiting examples, daratumumab (Darzalex) or any of the anti-CD38 antibodies having heavy and light chain variable sequences disclosed in FIG. 23, or any disclosed in U.S. Pat. Nos. 10,059,774, 9,951,144, or WO 2019/245616, all of which are incorporated by reference herein in their entireties. A PD-L1 antibody can be, as nonlimiting examples, durvalumab, pembrolizumab, atezolizumab, avelumab, or any of the anti-PD-L1 antibodies disclosed in U.S. Pat. No. 9,175,082, incorporated herein by reference.

Polypeptides of the present disclosure (e.g., antibodies and antibody fragments) can be produced using any methods known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

The recombinant DNA can also optionally encode any type of protein tag sequence that may be useful for purifying the protein. Examples of protein tags include but are not limited to a histidine (his) tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression vector construct can be introduced into a host cell, e.g., a production host cell, using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is a viral vector).

Suitable bacteria include gram negative or gram positive organisms, for example, E. coli or Bacillus spp. Yeast, for example from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, E. coli host cells are suitable for expressing small polypeptides. The protein can then be purified from culture media or cell extracts.

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system).

Nucleic acids encoding any of the various polypeptides disclosed herein may be synthesized chemically or using gene synthesis methods (available for example through commercial entities such as Blue Heron, DNA 2.0, GeneWiz, etc.). Codon usage may be selected so as to improve expression in a cell. Such codon usage will depend on the production host cell type. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified antibodies and antigen binding proteins described herein can be at least 65% pure, at least 75% pure, at least 85% pure, at least 95% pure, or at least 98% pure. Regardless of the exact numerical value of the purity, the polypeptide is sufficiently pure for use as a pharmaceutical product. Any of the anti-CD47 antibodies or tumor antigen-binding antibodies described herein can be expressed by transgenic host cells and then purified to about 65-98% purity or high level of purity using any art-known method.

In certain embodiments, the antibodies and antigen binding proteins herein can further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. In one embodiment, a form of glycosylation can be sialylation, which conjugates one or more sialic acid moieties to the polypeptide. Sialic acid moieties improve solubility and serum half-life while also reducing the possible immunogenicity of the protein. See Rajuetal. Biochemistry 2001 31; 40:8868-76.

In some embodiments, the antibodies and antigen binding proteins described herein can be modified to increase their solubility and/or serum half-life which comprises linking the antibodies and antigen binding proteins to non-proteinaceous polymers. For example, polyethylene glycol (“PEG”), polypropylene glycol, or polyoxyalkylenes can be conjugated to antigen-binding proteins, for example in the manner as set forth in U.S. Pat. Nos. 4,640,835; 4,496,689; 4,301,144; 4,670,417; 4,791,192; or 4,179,337.

The term “polyethylene glycol” or “PEG” is used broadly to encompass any polyethylene glycol molecule, without regard to size or to modification at an end of the PEG, and can be represented by the formula: X—O(CH₂CH₂O)ₙ—CH₂CH₂OH (1), where n is 20 to 2300 and X is H or a terminal modification, e.g., a C₁₋₄ alkyl. In one embodiment, the PEG terminates on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. Branched PEG molecules are described in, for example, EP-A 0 473 084 and U.S. Pat. No. 5,932,462. One form of PEGs includes two PEG side-chains (PEG2) linked via the primary amino groups of a lysine (Monfardini et al., Bioconjugate Chem. 6 (1995) 62-69).

### Pharmaceutical Compositions

The present disclosure provides pharmaceutical compositions comprising 1) any of the anti-CD47 antibodies described herein and 2) an antibody that specifically binds a tumor antigen, in a pharmaceutically acceptable excipient. The pharmaceutical compositions comprise an anti-CD47 antibody as disclosed herein, comprising a heavy chain variable region with an amino acid sequence having at least 95%, at least 96%, at least 97%, at least 98%, or at least 99% sequence identity to the amino acid sequence of SEQ ID NO:1 (the heavy chain variable region of antibody STI-6643) and an amino acid sequence having at least 95%, at least 96%, at least 97%, at least 98%, or at least 99% sequence identity to the amino acid sequence of SEQ ID NO:2 (the light chain variable region of antibody STI-6643). The antibody that specifically binds a tumor antigen can be any described herein, where the antibody includes an Fc region that can engage an Fcγ receptor.

The pharmaceutical compositions can be produced to be sterile and stable under the conditions of manufacture and storage. The antigen-binding proteins provided herein can be in powder form, for example for reconstitution in the appropriate pharmaceutically acceptable excipient before or at the time of delivery. Alternatively, the antigen-binding proteins can be in solution with an appropriate pharmaceutically acceptable excipient or a pharmaceutically acceptable excipient can be added and/or mixed before or at the time of delivery, for example to provide a unit dosage in injectable form. Preferably, the pharmaceutically acceptable excipient used in the present invention is suitable to high drug concentration, can maintain proper fluidity and, in some embodiments, can delay absorption.

Excipients encompass carriers and stabilizers. Examples of pharmaceutically acceptable excipients include for example inert diluents or fillers (e.g., sucrose and sorbitol), buffering agents, stabilizing agents, preservatives, non-ionic detergents, antioxidants, and isotonifiers. Depending on the type of formulation and the method of delivery, excipients can include lubricating agents, glidants, and anti-adhesives (e.g., magnesium stearate, zinc stearate, stearic acid, silicas, hydrogenated vegetable oils, or talc).

Therapeutic compositions and methods for preparing them are well known in the art and are found, for example, in “Remington: The Science and Practice of Pharmacy” (20th ed., ed. A. R. Gennaro A R., 2000, Lippincott Williams & Wilkins, Philadelphia, Pa.). Therapeutic compositions can be formulated for parenteral administration may, and can for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the antibodies (or an antigen binding protein thereof) described herein. Nanoparticulate formulations (e.g., biodegradable nanoparticles, solid lipid nanoparticles, liposomes) may be used to control the biodistribution of an antibody (or antigen binding protein thereof). Other potentially useful parenteral delivery systems include ethylene-vinyl acetate copolymer particles, osmotic pumps, implantable infusion systems, and liposomes. The concentration of an antibody (or antigen binding protein thereof) in the formulation varies depending upon a number of factors, including the dosage of the drug to be administered, and the route of administration.

Any of the anti-CD47 antibodies and anti-tumor antibodies as disclosed herein may be optionally administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like. In one example, the antibody (or antigen binding portions thereof) is formulated in the presence of sodium acetate to increase thermal stability.

Any of the anti-CD47 antibodies and anti-tumor antibodies as disclosed herein may be formulated for oral use include tablets containing the active ingredient(s) in a mixture with non-toxic pharmaceutically acceptable excipients. Formulations for oral use may also be provided as chewable tablets, or as hard gelatin capsules wherein the active ingredient is mixed with an inert solid diluent, or as soft gelatin capsules wherein the active ingredient is mixed with water or an oil medium.

Also provided is a kit comprising an anti-CD47 antibody as disclosed herein and an antibody that specifically binds a tumor antigen and includes an Fc region. The antibodies can be provided together, for example in a mixture, or may be provided in separate vials, ampules, packets, or other containers. The kit can further include one or more sterile pharmaceutically acceptable solutions for resuspension or dilution of one or both of the antibodies, and can include one or more additional pharmaceutical formulations, which may be, as nonlimiting examples, any of an additional antibody, an analgesic, or an antibiotic. The kit can be used for treating a subject having cancer. The components of the kit of can be provided in suitable containers and labeled for treatment of cancer. The above-mentioned components may be stored in unit or multi-dose containers, for example, sealed ampules, vials, bottles, syringes, and test tubes, as an aqueous, preferably sterile, solution or as a lyophilized, preferably sterile, formulation for reconstitution. The containers may be formed from a variety of materials such as glass or plastic and may have a sterile access port (for example, the container may be an intravenous solution bag or a vial having a stopper pierceable by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. Associated with the kits can be instructions customarily included in commercial packages of therapeutic, prophylactic or diagnostic products, that contain information about, for example, the indications, usage, dosage, manufacture, administration, contraindications and/or warnings concerning the use of such therapeutic, prophylactic or diagnostic products.

### Methods of Treatment

The present disclosure provides methods for treating a subject having a disease/disorder associated with expression or over-expression of one or more tumor-associated antigens. The disease comprises cancer or tumor cells expressing the tumor-associated antigens, such as for example CD38 or CD20 antigen. In one embodiment, the cancer or tumor includes cancer of the prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis and testis.

In various embodiments, the cancer comprises hematological cancers, including leukemias, lymphomas, myelomas, and B cell lymphomas. Hematologic cancers include multiple myeloma (MM), non-Hodgkin's lymphoma (NHL) including Burkitt's lymphoma (BL), B chronic lymphocytic leukemia (B-CLL), systemic lupus erythematosus (SLE), B and T acute lymphocytic leukemia (ALL), acute myeloid leukemia (AML), chronic lymphocytic leukemia (CLL), diffuse large B cell lymphoma, chronic myelogenous leukemia (CML), hairy cell leukemia (HCL), follicular lymphoma, Waldenstrom's Macroglobulinemia, mantle cell lymphoma, Hodgkin's Lymphoma (HL), plasma cell myeloma, precursor B cell lymphoblastic leukemia/lymphoma, plasmacytoma, giant cell myeloma, plasma cell myeloma, heavy-chain myeloma, light chain or Bence-Jones myeloma, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndroe, pemphigus vulgaris, scleroderma, multiple sclerosis, anti-phospholipid syndrome, ANCA associated vasculitis, Goodpasture's disease, Kawasaki disease, autoimmune hemolytic anemia, and rapidly progressive glomerulonephritis, heavy-chain disease, primary or immunocyte-associated amyloidosis, and monoclonal gammopathy of undetermined significance.

The methods include administering to the subject a therapeutically effective amount of a first antibody or an antigen binding fragment thereof that binds CD47 antigen and a second antibody that binds a tumor antigen, where the first antibody binds to CD47 antigen and blocks binding between CD47 antigen and SIRPα antigen, and wherein the second antibody binds a tumor cell and comprises Fc portion that binds an Fcγ receptor on an effector cell. The cancer can be a cancer that overexpresses CD47.

Also included are methods for killing at least one cancer cell in a population of cancer cells, wherein the at least one cancer cell overexpresses CD47 antigen, the method comprising: contacting the at least one cancer cell with a therapeutically effective amount of a first antibody or an antigen binding fragment thereof that binds CD47 antigen and a second antibody that binds a tumor antigen, where the first antibody binds to CD47 antigen and blocks binding between CD47 antigen and SIRPα antigen, and wherein the second antibody binds a tumor cell and comprises Fc portion that binds an Fcγ receptor on an effector cell.

The methods can use any of the CD47 antibodies disclosed herein, such as the STI-6643 antibody and variants thereof, and can use any tumor targeting antibodies, including but not limited to antibodies that specifically bind CD19, CD20, CD38, SLAMF7, or PD-L1, such as but not limited to those disclosed herein.

In some embodiments, treatment of a subject with cancer with a combination of the CD47 antibody provided herein in addition to a tumor targeting antibody, such as an anti-CD38 or anti-CD20 antibody can have a synergistic effect with respect to treatment of a subject with cancer with only the tumor targeting antibody or only the CD47 antibody. The synergistic effects can be reduction in tumor volume or increased survivorship, as nonlimiting examples.

In some embodiments, treatment of a subject with cancer with a combination of a tumor targeting antibody and a CD47 antibody as provided herein, i.e., STI-6643, or an antibody having a heavy chain variable region having at least 95%, at least 96%, at least 97%, at least 98%, or at least 99% identity to SEQ ID NO:1 and a light chain variable region having at least 95%, at least 96%, at least 97%, at least 98%, or at least 99% identity to SEQ ID NO:2 can result in less toxicity to the subject, including, without limitation, less anemia, sustained hemoglobin concentration, reduced hemagglutination of red blood cells and/or reduction of healthy immune cells, than treatment of a patient with the same tumor targeting antibody and a different anti-CD47 antibody.

In some embodiments, administration of the antibody that specifically binds CD47 can be by oral delivery. Oral dosage forms can be formulated for example as tablets, troches, lozenges, aqueous or oily suspensions, dispersible powders or granules, emulsions, hard capsules, soft gelatin capsules, syrups or elixirs, pills, dragees, liquids, gels, or slurries. These formulations can include pharmaceutically excipients including, but not limited to, inert diluents such as calcium carbonate, sodium carbonate, lactose, calcium phosphate or sodium phosphate; granulating and disintegrating agents such as corn starch or alginic acid; binding agents such as starch, gelatin or acacia; lubricating agents such as calcium stearate, glyceryl behenate, hydrogenated vegetable oils, magnesium stearate, mineral oil, polyethylene glycol, sodium stearyl, fumarate, stearic acid, talc, zinc stearate; preservatives such as n-propyl-p-hydroxybenzoate; coloring, flavoring or sweetening agents such as sucrose, saccharine, glycerol, propylene glycol or sorbitol; vegetable oils such as arachis oil, olive oil, sesame oil or coconut oil; mineral oils such as liquid paraffin; wetting agents such as benzalkonium chloride, docusate sodium, lecithin, poloxamer, sodium lauryl sulfate, sorbitan esters; and thickening agents such as agar, alginic acid, beeswax, carboxymethyl cellulose calcium, carageenan, dextrin or gelatin.

In various embodiments, administration can be by injection or intravenous or intra-arterial delivery, and may be, for example, by epidermal, intradermal, subcutaneous, intramuscular, intraperitoneal, intrapleural, intra-abdominal, or intracavitary delivery. Formulations for parenteral administration can be inter alia in the form of aqueous or non-aqueous isotonic sterile non-toxic injection or infusion solutions or suspensions. Preferred parenteral administration routes include intravenous, intra-arterial, intraperitoneal, epidural, and intramuscular injection or infusion. The solutions or suspensions may comprise agents that are non-toxic to recipients at the dosages and concentrations employed such as 1,3-butanediol, Ringer's solution, Hank's solution, isotonic sodium chloride solution, oils such as synthetic mono- or diglycerides or fatty acids such as oleic acid, local anesthetic agents, preservatives, buffers, viscosity or solubility increasing agents, water-soluble antioxidants such as ascorbic acid, cysteine hydrochloride, sodium bisulfate, sodium metabisulfite, sodium sulfite and the like, oil-soluble antioxidants such as ascorbyl palmitate, butylated hydroxyanisole (BHA), butylated hydroxytoluene (BHT), lecithin, propyl gallate, alpha-tocopherol, and the like, and metal chelating agents, such as citric acid, ethylenediamine tetraacetic acid (EDTA), sorbitol, tartaric acid, phosphoric acid, etc.

In some embodiments, the methods result in lower toxicities to the patient than treatment with other antibodies that exhibit a higher degree of binding to red blood cells.

### EXAMPLES

The following examples are meant to be illustrative and can be used to further understand embodiments of the present disclosure and should not be construed as limiting the scope of the present teachings in any way.

### Example 1. STI-6643 Demonstrates Drastically Reduced Hemagglutination as Compared to Competitor Antibody Hu5F9

Preparation of Red Blood Cells (RBCs): Peripheral blood was obtained from healthy human donors. 4 mL of blood was pipetted into a 15 mL conical tube and topped off with 1× PBS at room temperature (RT). Cells were centrifuged at 800 rpm for 10 minutes. The supernatant was aspirated without disturbing the RBCs at the bottom of the tubes and 12 ml of 1× PBS were added. The cells were mixed by inverting the tube. The cells were centrifuged at 800 rpm for 5 minutes and the wash was repeated twice. The supernatant was aspirated after the final wash without disturbing the blood cells and enough 1× PBS was added to make a 10% solution of RBCs (this solution was useable for 1 week). To make a final working solution the 10% solution pf RBCs in 1X PBS was diluted to obtain a 0.5% solution.

For the hemagglutination assay, 0.5% RBCs working solution was mixed by inverting the tube. 0.5% RBCs working solution was added to each well of a U-bottom 96-well plate RT (50 μL). The following antibodies were used in this assay: STI-6643, anti-CD47 IgG4 Hu5F9, and isotype IgG4 control. Serial dilutions of antibodies were prepared in an ultra-low attachment 96-well plate in 1× PBS (2-fold dilutions starting at 75 μg/mL). The antibody dilutions (100 μL/well) were transferred into the plate containing RBCs (final starting antibody concentration: 50 μg/mL) and mixed slowly a few times with a multichannel pipet. The plate was placed into a tissue culture incubator (5% CO₂, 37° C.) for a ˜20 h incubation. Negative results (no hemagglutination) appear as red dots in the centre of round-bottomed plates. Positive results (hemagglutination) will form a uniform reddish color across the well.

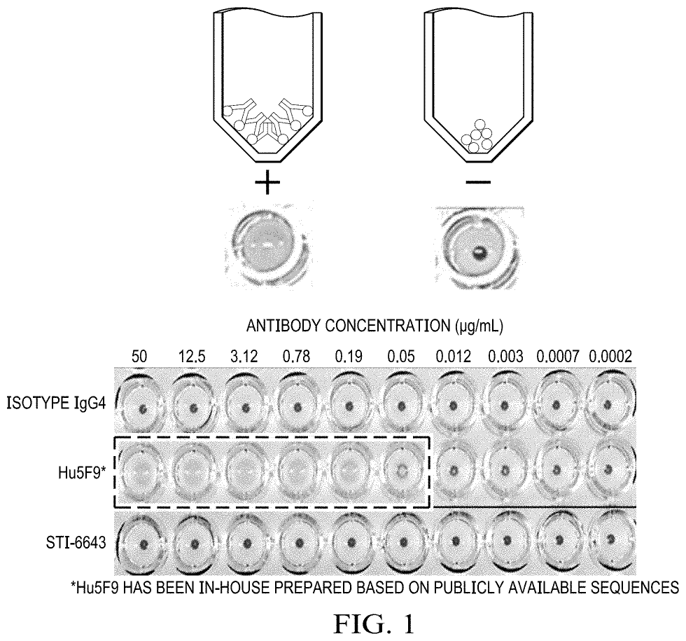

FIG. 1 provides a diagram of agglutination and lack of agglutination over the visible appearance of corresponding wells. Antibody Hu5F9 shows agglutination in the wells corresponding to antibody concentrations of from 50 μg/mL to 0.05 μg/mL, with agglutination disappearing at antibody concentration of 0.012 μg/mL and below. In contrast, even at the highest antibody concentration of STI-6643 (50 μg/mL), agglutination is not apparent.

### Example 2. STI-6643 Blocks Human CD47/SIRPα Interaction in a Dose-Dependent Manner

To determine whether binding of STI-6643 to cells of T lymphoblastic leukemia cell line CCRF-CEM could block binding of cells to SIRPα (receptor for CD47), CCRF-CEM cells (20,000 cells in 50 μL/well) were incubated with either anti-CD47 (clones STI-6643 or Hu5F9) or isotype IgG4 control antibodies at concentrations ranging from 400 to 0.18 μg/mL in FACS buffer (1× PBS+2% FCS) and incubated for 15 minutes at 37° C. Without washing, purified SIRPα-Fc fusion protein (R&D system; Cat#4546-SA-050) was added to each well at a concentration of 0.4 μg/mL (50 μL/well) in FACS buffer (maintained at 37° C.) and the incubation was continued for another 20 minutes at 37° C. Then, CCRF-CEM cells were washed thrice by centrifugation at 524 g for 2 minutes at room temperature (RT) and resuspended each time in 170 μL/well of RT FACS buffer. To reveal the binding of SIRPα-Fc fusion protein to CCRF-CEM cells, a PE-labelled anti-SIRPα antibody (R&D system; Cat#FAB4546P) was used at 5 μL/well in 70 μL of RT FACS buffer. The cells were incubated for 20 minutes at RT in the dark. Then, CCRF-CEM cells were washed twice by centrifugation at 524 g for 2 minutes at room temperature (RT) and resuspended each time in 170 μL/well of RT FACS buffer. Samples were immediately acquired by flow cytometry and analyzed by using FlowJo.

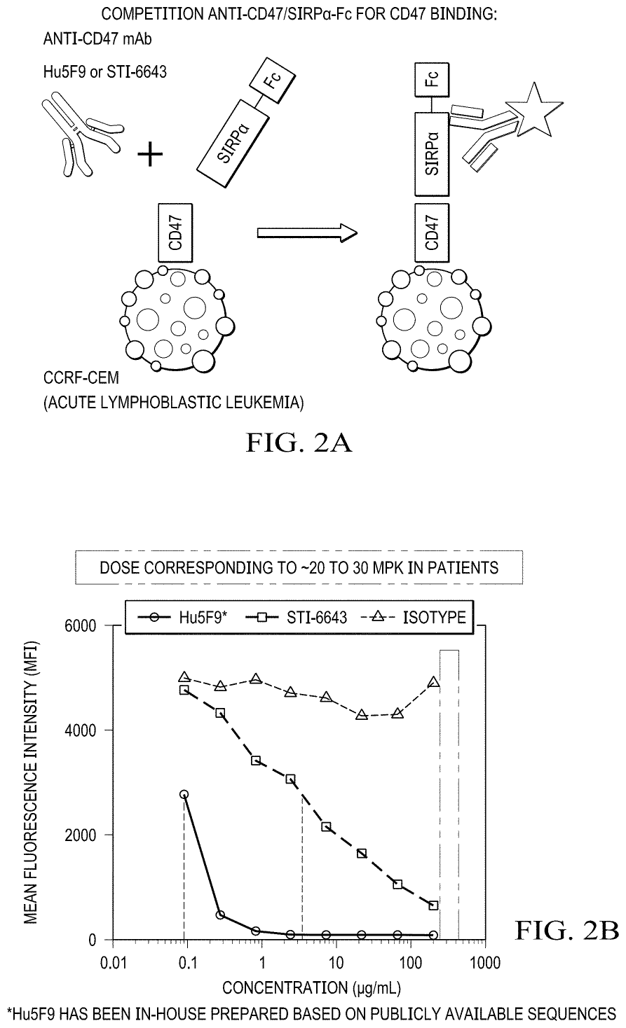

FIG. 2B shows that addition of the IgG4 isotype control antibody has no effect on binding of SIRPα to the CCRF-CEM cells (upper curve demonstrating no dose dependence). On the other hand, there is a strong dose dependence of STI-6643 when it is added to the CCRF-CEM cells, with a strong decrease in binding of SIRPα to the CCRF-CEM cells with increasing concentrations of STI-6643 antibody (curve descending diagonally across the graph from MFI>4,000 to less than 1,000. The Hu5F9 antibody strongly inhibits binding of SIRPα to CCRF-CEM cells at antibody concentrations of 1 μg/mL and above.

### Example 3. STI-6643 Promotes Phagocytosis of Tumor Cells in a Dose-Dependent Manner

10,000 RAJI-GFP cells in 50 μL of RPMI 1640 supplemented with 10% FBS and antibiotics at room temperature (RT) were transferred into a flat bottom 96 well plate. Antibodies (STI-6643, Hu5F9, and isotype IgG4) were serially diluted starting from concentration of 400 μg/mL. Antibody dilutions (50 μL) were added to the wells containing the tumor cells.

For the ADCP assay, peripheral blood obtained from two healthy human donors was used as the source for PBMCs (containing CD14⁺ phagocytic cells). 30,000 PBMCs in 100 μL were added in each well for a 3:1 ratio of PBMCs to Raji cells in each well. The wells were mixed and spun for 1 minute at 1,500 rpm and the wells were incubated for 90 minutes at 37° C. before harvesting for analysis by flow cytometry.

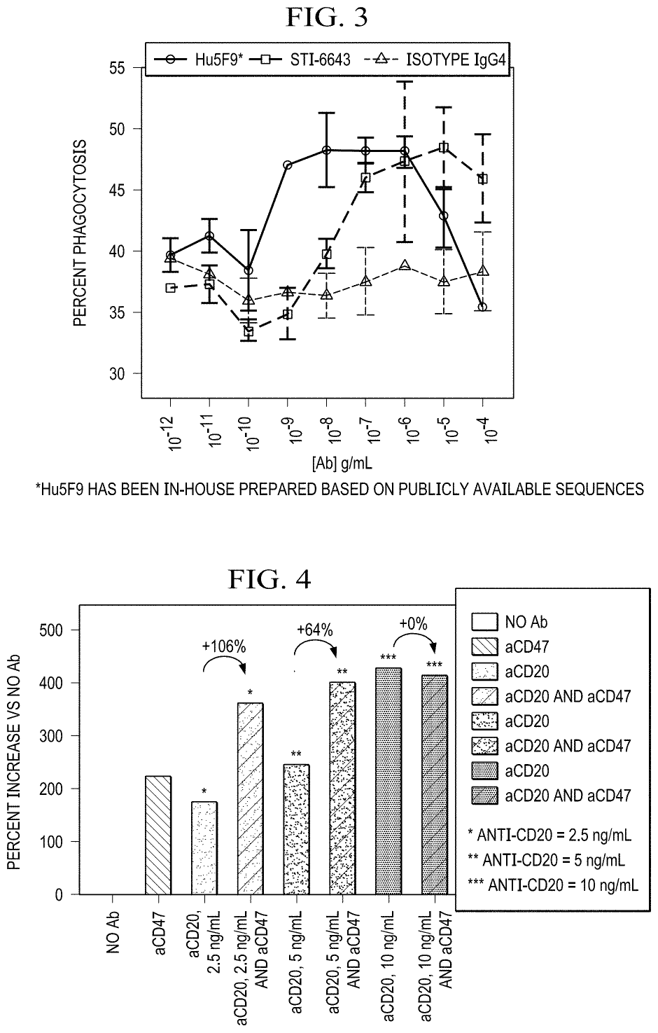

Nonadherent cells were transferred to a 96-well V bottom plate, the plate was spun 3 minutes at 1,500 rpm and the cells were resuspended in 100 μl FACS Buffer at 4° C. (PBS 2% FBS, 2 mM EDTA). 100 μL Tryple (Thermo Fisher, cat. 12604013) was added to the first plate to detach the remaining nonadherent cells, and those cells were transferred to the V bottom plate containing nonadherent cells. After centrifuging 3 minutes at 1,500 rpm and resuspending in 100 μL of staining mix containing anti-CD14-PE (1.5 μL/well in 90 μL) diluted in FACS Buffer, the cells were incubated for 15 minutes at 4° C. and then washed once with 100 μL FACS Buffer. The cells were then resuspended in 100 μL of Fixation Buffer (Biolegend, cat. 420801) for 20 minutes at 4° C. 100 μL FACS Buffer was directly added (final volume 200 μL) and run on a flow cytometer. Data was analyzed by using the FlowJo Software. FIG. 3 shows that phagocytosis in the presence of the Isotype IgG4 did not increase in a dose dependent manner, remaining at between about 35% and 40%. When antibody STI-6643 was used in the assay, a large increase in % phagocytosis was observed as the concentration of antibody increased, beginning at an antibody concentration of approximately 10⁻⁹. The Hu5F9 antibody also showed dose-dependence, with a rapid rise in phagocytosis observed beginning at an antibody concentration of approximately 10⁻¹⁰ and leveling off at a concentration of about 10⁻⁸.

### Example 4. STI-6643 Improves Rituximab-induced Phagocytosis When Combined at Suboptimal Doses

CD14+ cells were isolated from human PBMCs and differentiated into macrophages by culturing the cells in RPMI-1640 supplemented with 20% FBS, antibiotics and 20 ng/mL M-CSF. Cells were plated in a flat bottom 96-well plate (30,000 cells in 100 μL per well) and incubated for 7 days or until differentiation is observed. Medium was refreshed every 2-3 days with complete medium.

60,000 RAJI-GFP cells in 50 μL of RPMI 1640 supplemented with 10% FBS and antibiotics at room temperature (RT) were transferred into a U-bottom 96 well plate. Antibodies were serially diluted with a final dilution of 10 μg/mL for STI-6643 and 2.5, 5, or 10 ng/mL for Rituximab. 50 μL of antibody was added to the wells containing tumor cells.

The ADCP assay was initiated by transferring 100 μL of the RAJI-antibody mixture on top of the human macrophages in the flat bottom 96-well plate. The cells and antibodies were mixed and centrifuged for 1 minute at 1,500 rpm, after which the plate was incubated for 30 minutes at 37° C. before harvesting for analysis by flow cytometry. After 20 minutes 1 μL of anti-CD11 b-PE was added to each well.

For flow cytometry, nonadherent cells were transferred into a 96-well V-bottom plate, centrifuged for 3 minutes at 1,500 rpm and resuspended in 100 μl FACS Buffer at 4° C. (PBS 2% FBS 2 mM EDTA). Accutase (150 μL) (Fisher Scientific, cat. NC9839010) was added to the flat bottom plate to detach the remaining nonadherent cells, the cells were pipeted and transferred to the V bottom plate containing nonadherent cells. The cells were centrifuged 5 minutes at 1,500 rpm and resuspended in 150 μL/well of stabilizing fixative (Fisher Scientific, cat. 338036). Data were acquired on a flow cytometer and analyzed by using the FlowJo Software.

FIG. 4 shows that the anti-CD20 antibody rituximab (solid colored bars) and the anti-CD47 antibody (solid black bar) when administered separately each induce phagocytosis. When the anti-CD20 antibody and the anti-CD47 antibody are administered together (bars with black diagonals), there is an enhancement of phagocytosis with respect to the use of anti-CD20 antibody alone. At the lowest concentration of anti-CD20 antibody, the enhancement of phagocytosis when STI-6643 is included is greatest, resulting in approximately double the phagocytosis observed when anti-CD20 is used on its own.

### Example 5. STI-6643 Shows Comparable Anti-Tumor Activity Than Hu5F9 in a Preclinical Mouse Model

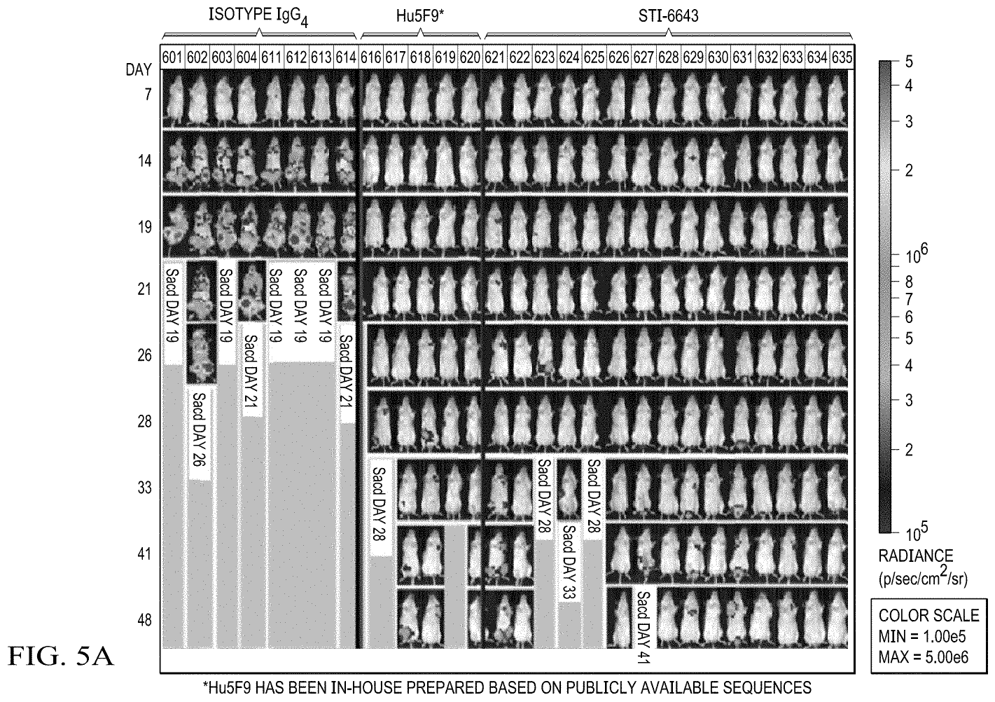

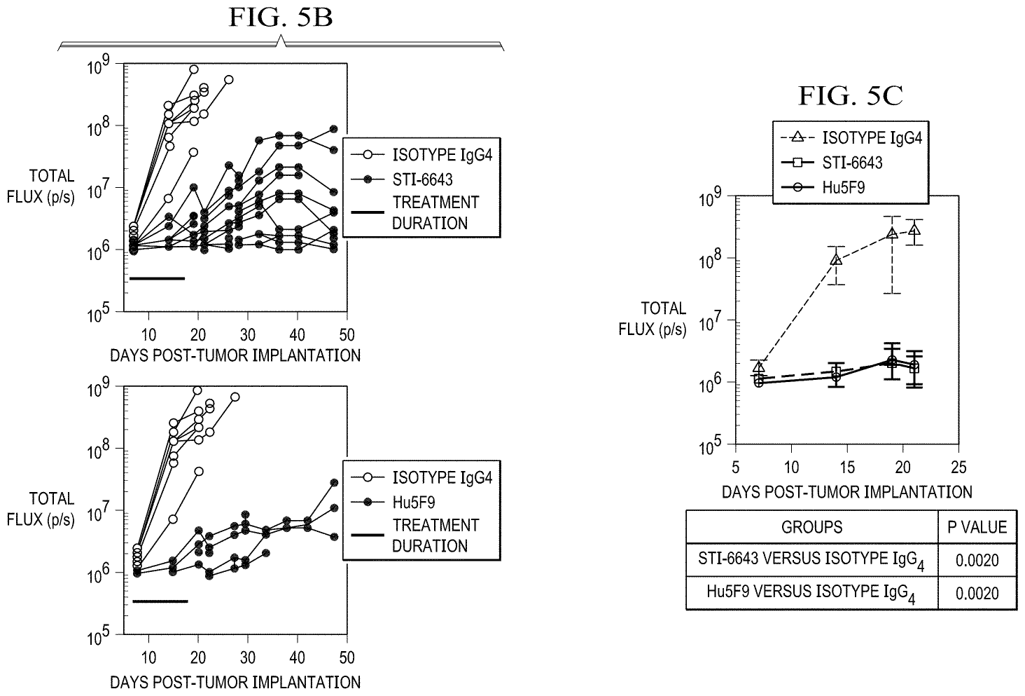

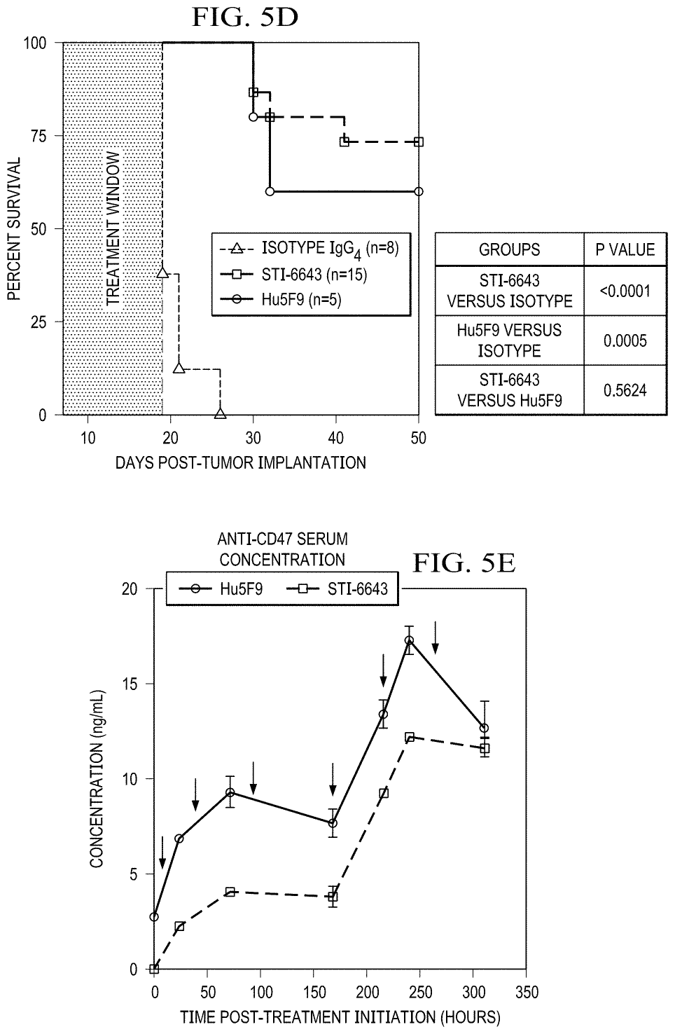

Fox Chase SCID mice (n=8 for Isotype IgG₄; n=5 for Hu5F9 and n=15 for STI-6643 group) transplanted intravenously with luciferase expressing Raji cells (RAJI-Fluc) were treated with 30 mg/kg of the indicated antibodies three times a week for 2 consecutive weeks starting on day 7 post RAJI-Fluc tumor inoculation. Luciferase imaging of representative mice from day 7 to 48 post tumor cells inoculation (FIG. 5A) and luminescence data for individual mice (FIG. 5B) or mean +/−SD of total flux values plotted only until d22, before survival declines (FIG. 5C) are shown. Statistical significance was assessed by 2-way ANOVA where each row represents a different time point (so match values are spread across a row), comparing column means (main column effect) and corrected for multiple comparisons using the Tukey's comparisons test, with individual variances computed for each comparison. Kaplan-Meier survival analysis was performed (FIG. 5D). The p values comparing each group with the other two groups are shown (p<0.05 is considered statistically significant). The interval of time during which mice received treatment is represented by a grey area. Circulating antibody concentration was evaluated in each animal treated with anti-CD47 (STI-6643 or Hu5F9) from day 0 to 13 post treatment initiation (FIG. 5E). Data are given as a mean +/−S.D. Arrows represent the time of antibody treatments for each group. All statistical tests were 2-sided, and results were considered statistically significant at P<0.05.

### Example 6. Combination of STI-6643 and Rituximab Improves Anti-Tumor Activity and Prolonged Survival

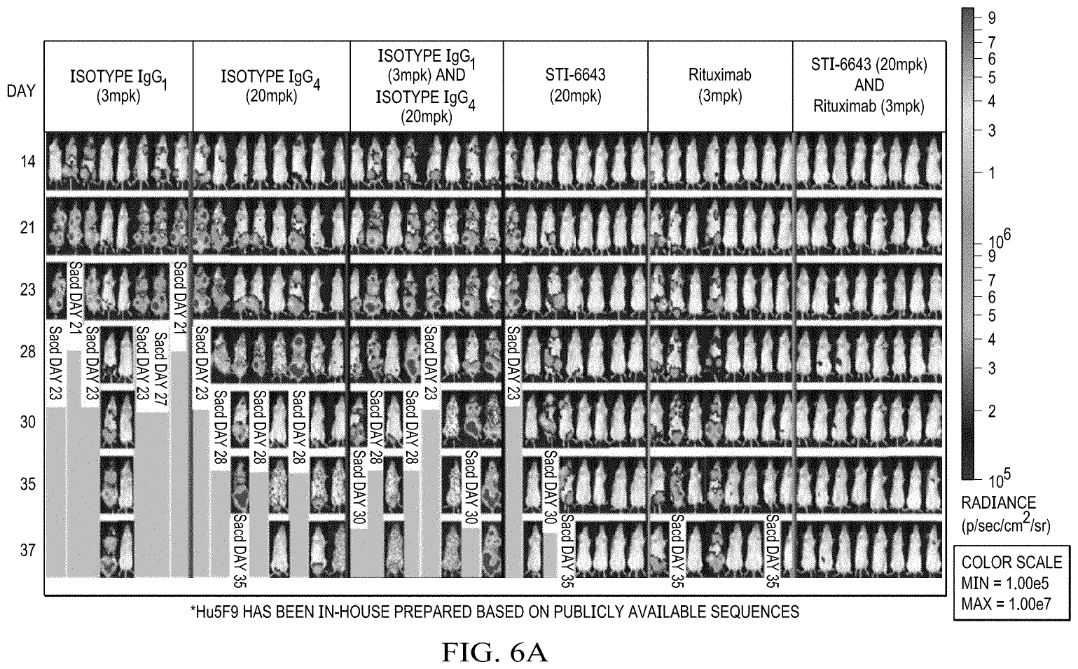

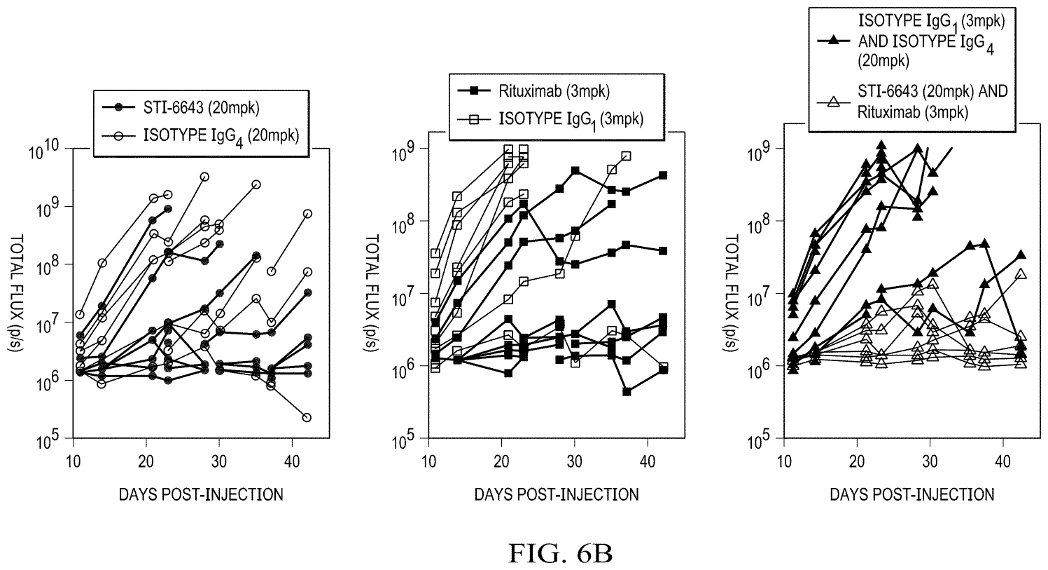

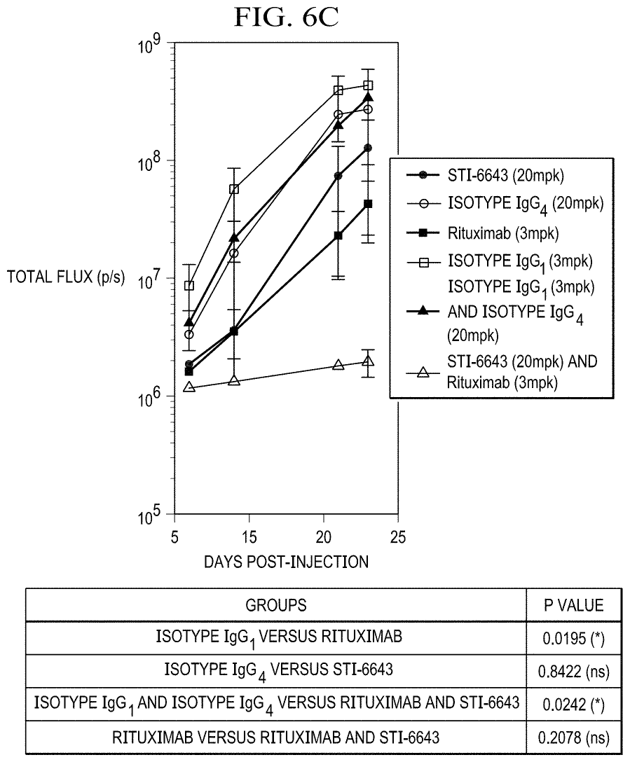

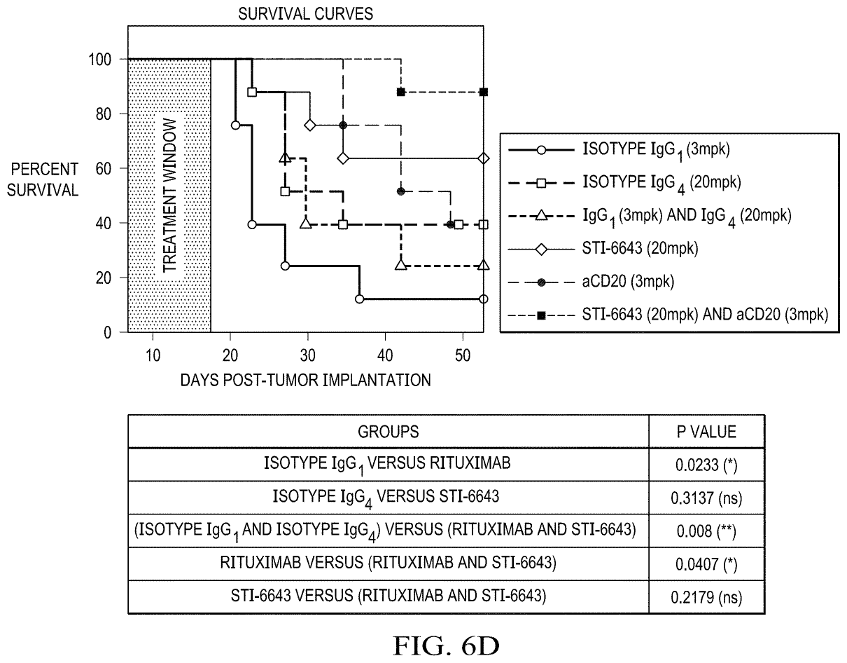

Combination Therapy with Anti-CD47 Antibody and Rituximab Eliminates Lymphoma in Disseminated Human RAJI xenograft Mouse Models. Fox Chase SCID mice (n=8 per group) transplanted intravenously with luciferase expressing Raji cells (RAJI-Fluc) were treated with 3 mg/kg of Rituximab (anti-CD20 antibody) or its IgG₁ isotype control or 20 mg/kg of STI-6643 or its IgG₄ isotype control antibodies alone or in combination as indicated. Treatment was given three times a week for 2 consecutive weeks starting on day 7 post RAJI-Fluc inoculation (day 7, 9, 11, 14, 16 and 18). Luciferase imaging of representative mice from day 14 to 37 post tumor cells inoculation (FIG. 6A). Luminescence data for individual mice (FIG. 6B) or mean +/−SEM of total flux values plotted only until d23, before survival declines (FIG. 6C) are shown. Statistical significance was assessed by 2-way ANOVA followed by Tukey's multiple comparison test. Kaplan-Meier survival analysis was performed (FIG. 6D). p values comparing each group with the others are shown. The interval of time during which mice received treatment is represented by a grey area. All statistical tests were 2-sided, and results were considered statistically significant at P<0.05. Treatment of mice with STI-6643 in combination with anti-CD20 had the greatest degree of survival (top line), followed by treatment with STI-6643 alone, and then by anti-CD20 alone. The increase in survival between mice treated with Rituximab (anti-CD20 antibody) plus STI-6643 (anti-CD47 antibody) and Rituximab below was statistically significant.

### Example 7. STI-6643 is Well Tolerated and Safe in Non-Human Primates at 150 mg/kg

The objective of this Non-GLP dose-finding Toxicity study was to determine the potential toxicity of STI-6643 when given by intravenous (IV) bolus injection once weekly (on Days 1, 8, 15, and 22) for a total of 4 doses to cynomolgus monkeys without any priming dose (as shown in Table 1). Animals underwent a 28-day recovery period before being necropsied on Day 57. The study design was as follows:

TABLE 1

| | | | Dose | Dose | No. of Animals^{a} | |
|---|---|---|---|---|---|---|
| Group | Test | Dose | volume | Conc'n. | No. | No. |
| No. | material | (mg/kg) | (mL/kg) | (mg/mL) | males | females |
| 1 | Control | 0 (vehicle) | 5 | 0 (vehicle) | 2 | 2 |
| 2 | STI-6643 | 30 | 5 | 30 | 2 | 2 |
| 3 | STI-6643 | 90 | 5 | 90 | 2 | 2 |
| 4 | STI-6643 | 150 | 5 | 150 | 2 | 2 |

Groups 1 and 2 were dosed first, Group 3 commenced dosing approximately 2 weeks after Groups 1 and 2; Group 4 commenced dosing approximately 5 weeks after Group 3

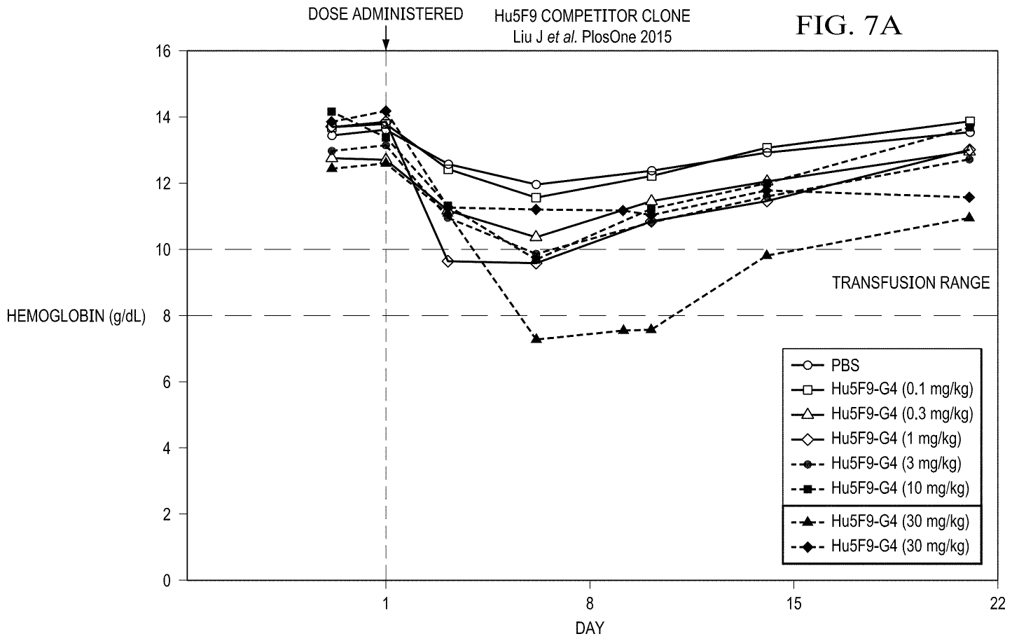

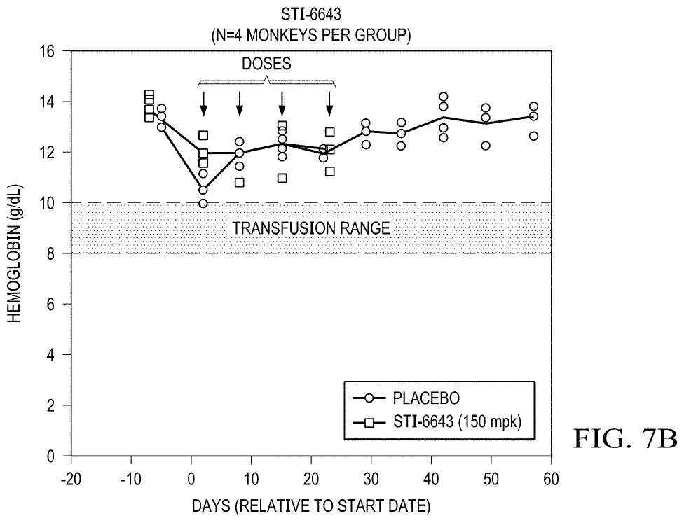

All animals^{a} underwent necropsy on day 57. A graph showing the circulating hemoglobin concentration over time is shown in FIG. 7B.

The following parameters and end points were evaluated in this study: clinical observations (cage side, post-dose, and detailed), body weights, qualitative food consumption, clinical pathology parameters (hematology, coagulation, and clinical chemistry), bone marrow evaluation, bioanalysis, anti-drug antibodies, toxicokinetic parameters, gross necropsy observations, organ weights, and histopathologic examinations. All animals survived to scheduled necropsy.

Since anemia is known to be one of the major adverse events upon anti-CD47 treatment, we decided to compare the hemoglobin level over time when cynomolgus monkeys where treated with a single dose of Hu5F9 as published by Liu J et al. (Plos One, 2015) (FIG. 7A) or four consecutive doses of STI-6643 (FIG. 7B).

Conclusion: STI-6643-related changes in clinical chemistry parameters were limited to non-adverse, slightly decreased urea nitrogen on Day 29 at 150 mg/kg/dose. The Day 29 urea nitrogen at 150 mg/kg/dose was also statistically significantly decreased compared to control values.

There were no STI-6643-related changes in clinical observations, body weights, qualitative food consumption, hematology, coagulation parameters, bone marrow evaluation, gross necropsy observations, organ weights, or histopathology. In conclusion, administration of STI-6643 by intravenous bolus injection once weekly (on Days 1, 8, 15, and 22) for a total of 4 doses was well-tolerated in cynomolgus monkeys at levels up to 150 mg/kg/dose. Based on these results, the no-observed-adverse-effect level (NOAEL) was considered to be 150 mg/kg/dose for up to four doses.

### Example 8. Anti-CD47 Clone STI-6643 Shows Preferential Binding to Tumor Cells. Binding Assay on Mixed-Cell Samples

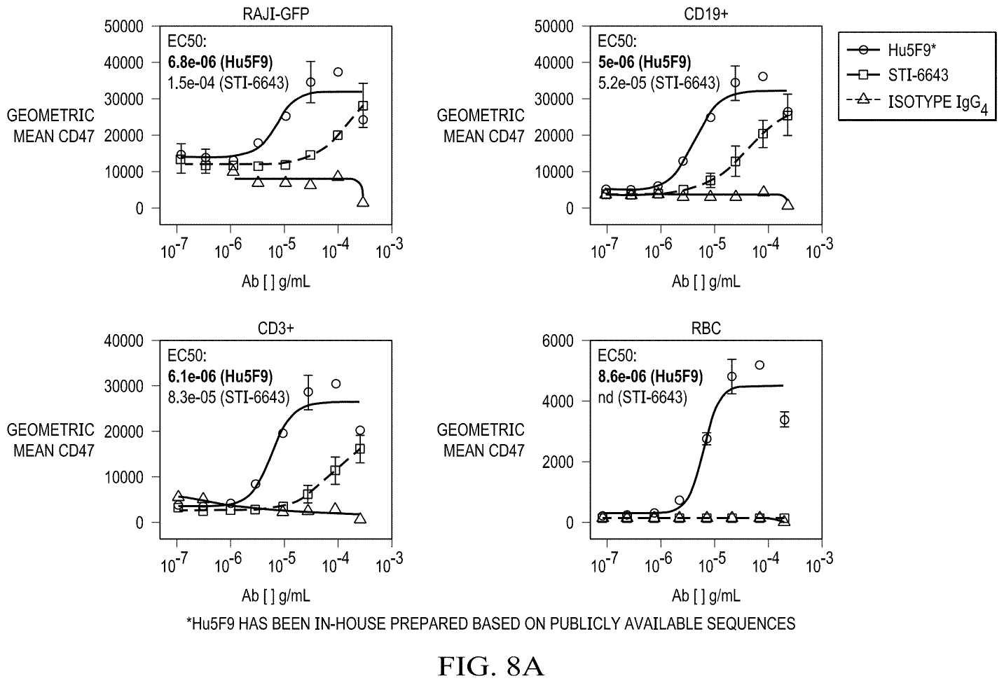

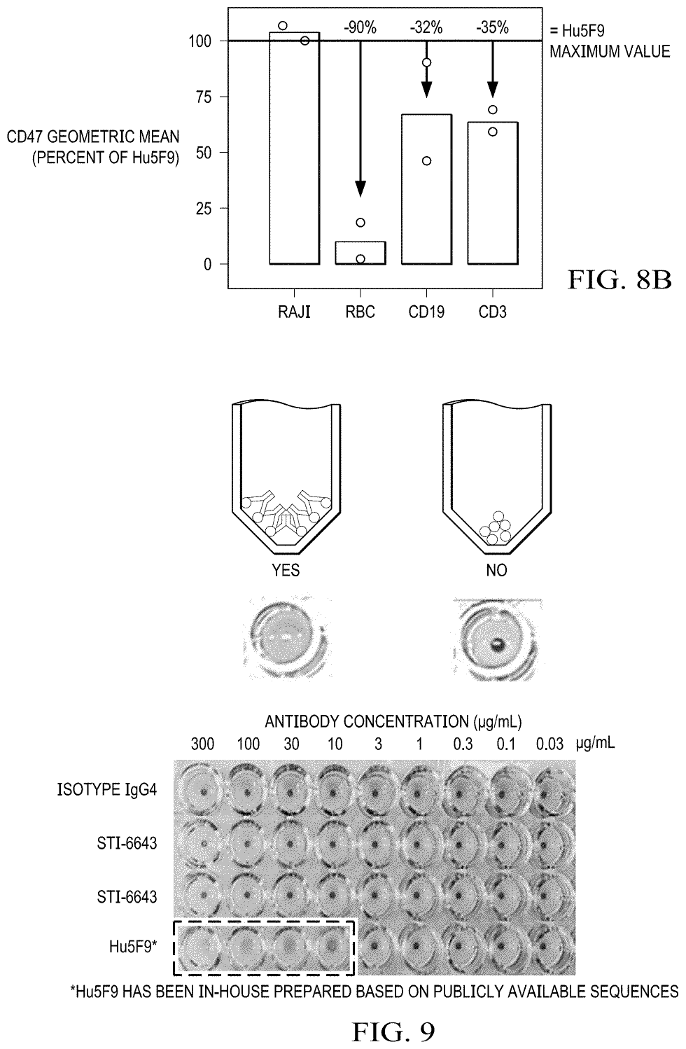

Human whole blood (25 μL/well) and RAJI-GFP (5,000 cells per well) were mixed and stained with various concentrations (from 300 μg/mL to 1 ng/mL) of anti-CD47 (STI-6643 or competitor Hu5F9 expressed in-house) or Isotype IgG4 control antibodies for 45 minutes at 37° C. Cells were washed twice then incubated with the following antibody mixture: Secondary antibody (APC-labeled anti-human-Fc), anti-CD45-BV711, anti-CD3-BV510, anti-CD19-APC-Cy7 and anti-CD235a-PB for 20 minutes at 37° C. After two washes, cells were fixed and analyzed by flow cytometry. FIG. 8A shows that the Hu5F9 antibody (upper curve in each graph) binds both Raji tumor cells and RBCs with EC₅₀ₛ of 5.1×10⁻⁶ and 3.3.×10⁻⁶, respectively, while the binding of Raji tumor cells and RBCs by anti-CD47 antibody STI-6643 was found to have an EC of approximately 6.4×10⁻⁵ and 5.6×10⁻⁵, respectively. For both Raji tumor cells and red blood cells, specific binding begins to occur at a concentration of about 10⁻⁵ g/ml; however binding of Raji tumor cells by the STI-6643 antibody rises to the level of binding demonstrated by antibody Hu5F9 at a concentration of approximately 5×10⁻³ g/ml, whereas binding of RBCs by the STI-6643 antibody remains very low with respect to the binding of RBCs by the Hu5F9 antibody.

The percentage of binding of STI-6643 to RAJI tumor cell, red blood cells (RBC), B (CD19+) or T (CD3+) cells was evaluated at the highest dose (300 μg/mL) as compared to a relative 100% binding given by the Hu5F9 clone at the same antibody concentration (as calculated by the geometric mean fluorescence intensity) is shown in the bar graph in FIG. 8B.

Conclusion: STI-6643 anti-CD47 antibody bound Raji tumor cells at least as well as the Hu5F9 antibody bound Raji cells, whereas the binding of STI-6643 to RBCs was only approximately 10% of the binding to RBCs exhibited by the Hu5F9 antibody.

### Example 9. STI-6643 Drastically Reduces Hemagglutination as Compared to Competitor Antibody Hu5F9 (Additional Hemagglutination Experiment)

To prepare red blood cells (RBCs), peripheral blood was obtained from a healthy human donor. 4 mL of blood was pipetted into a 15 mL conical tube and topped off with 1× PBS at room temperature (RT). The tube was spun at 140 g for 10 minutes. The supernatant was aspirated without disturbing the RBCs at the bottom of the tubes. 12 mL of 1× PBS was added and mixed by inverting the tube. The cells were centrifuged at 140 g for 5 minutes and the wash was repeated twice. The supernatant was aspirated after the final wash without disturbing the RBCs and enough 1× PBS was added to make a 10% solution of RBCs. To make a final working solution the 10% solution RBCs was diluted in 1× PBS to obtain a 0.5% solution.

For the hemagglutination assay, 50 μL of 0.5% RBCs working solution were added to each well of a U-bottom 96-well plate and the plate was maintained at RT. The following antibodies were used in this assay: anti-CD47 IgG4 (clones STI-6643 and Hu5F9) and isotype IgG4 control. Serial dilutions of antibodies were made in an ultra-low attachment 96-well plate in 1× PBS (2-fold dilutions starting at 450 μg/mL). The antibody dilutions were transferred into the plate containing RBCs (100 μL/well, starting antibody concentration: 300 μg/mL) and slowly mixed a few times with a multichannel pipet. The plate was placed in a tissue culture incubator (5% CO_{2,} 37° C.) for a ˜20 h incubation. Negative results (no hemagglutination) appeared as dots in the centre of round-bottomed plates. Positive results (hemagglutination) formed a uniform reddish color across the well. FIG. 9 (top) provides a diagram and example of positive and negative results. FIG. 9 (bottom) shows the results of the assay.

Conclusion: STI-6643 does not induce hemagglutination even at high concentration.

### Example 10: STI-6643 Preserves the Adaptive and Innate Immune System: 3-Way MLR Assay

On day 0, peripheral blood mononuclear cells (PBMCs) from three different human healthy donors were prepared and resuspended into complete RPMI-10AB medium (RPMI1640 supplemented with 10% human AB serum from Valley Biomedical, ref HP1022, lot 6F1131). An equal number of PBMCs from each donor was plated on a flat-bottom 96-well plate to obtain a 1.65E+05 cells/donor/well (˜5.0E+05 cells/well final) seeding density in 100 μL of RPMI-10AB. Isotype control or anti-CD47 (STI-6643 or Hu5F9) human IgG₄ antibodies were diluted in complete RPMI-10AB medium at a 2× concentration (10-fold serial dilutions starting at 200 μg/mL), then subsequently 100 μL/well was added to the appropriate wells for a final concentration ranging from 100 μg/mL to 1 ng/mL. The plate was incubated for 6 days in a humidified tissue culture incubator (37° C., 5% CO₂).

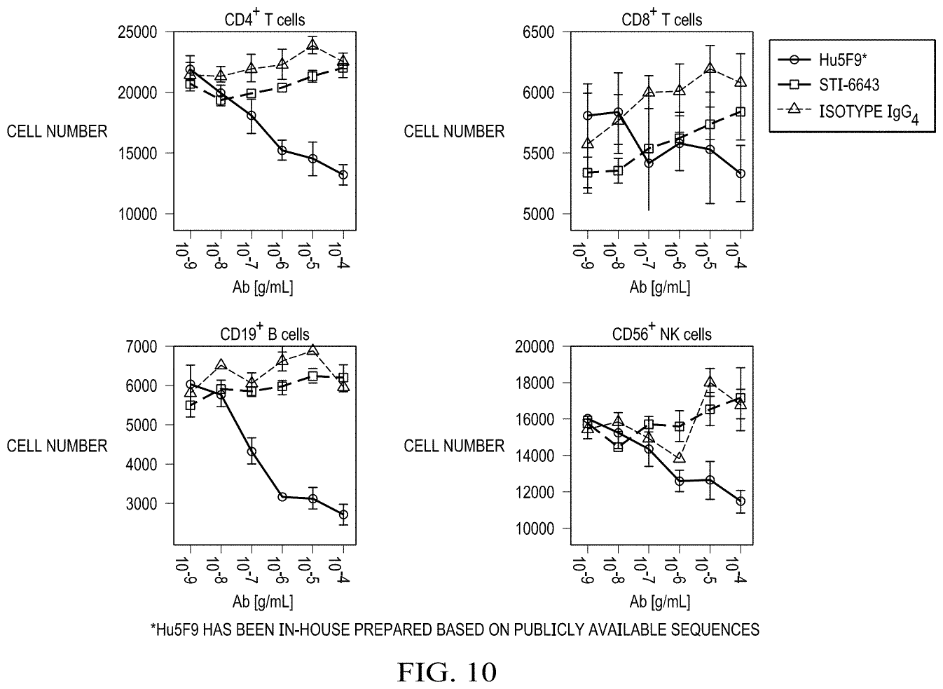

On day 6 post-co-culture, the cells were spun at 300 g for 5 minutes and washed using cold FACS buffer (Dulbecco's Phosphate Buffered Saline supplemented with 2 mM EDTA and 2% Fetal Bovine Serum). Then, cells were incubated for 30 minutes at 4° C. with an antibody mixture composed of PE-conjugated CD4 (clone OKT4; Biolegend, Cat. no. 317410, lot# B264363), FITC-conjugated CD8 (clone HIT8a; Biolegend, Cat. no. 300906, lot# B275277), APC-Cy7-conjugated CD19 (clone SJ25C1; Biolegend, Cat. no. 363010, lot# B276795), Pacific Blue-conjugated CD56 (clone HCD56; Biolegend, Cat. no. 318326, lot# B280451). After washing cells twice with 150 μL/well of FACS buffer, they were fixed using 100 μL/well of fixation buffer (Biolegend; Cat. 420801) for 20 minutes at room temperature. Subsequently, cells were washed once with 150 μL/well of FACS buffer and the number of CD4⁺, CD8⁺, CD19⁺ and CD56⁺ cells was measured by flow cytometry and analyzed using the FlowJo software. Data represent the mean +/−S.E.M of triplicate values for each point. The results of CD4⁺, CD8⁺, CD19⁺ and CD56⁺ are shown in FIG. 10.

Conclusion: STI-6643 sustains T, B and NK cells viability in an MLR-induced proinflammatory milieu.

### Example 11: STI-6643 Better Preserves T Cells Number and Activation in a SEB Assay

Fresh PBMCs were isolated and diluted at 2.0E+06 cells/mL in complete RPMI (RPMI 1640+10% FCS+Pen/Strep). Cells were plated out at 2.0E+05 cells/well in a in U-bottom plate (100 μL/well). Next, anti-CD47 (STI-6643 or Hu5F9) or isotype control antibody clones were serially diluted (from 100 μg/mL to 1 ng/mL) in complete RPMI containing SEB (Staphylococcal Enterotoxin B from List Biological laboratories; Cat. no. 122, lot# 1224171) at 100 ng/mL final concentration (50 4 of antibody preparation mixed with 50 μL of SEB both at 4× concentration). The plates were placed in a 37° C. incubator for 3 days.

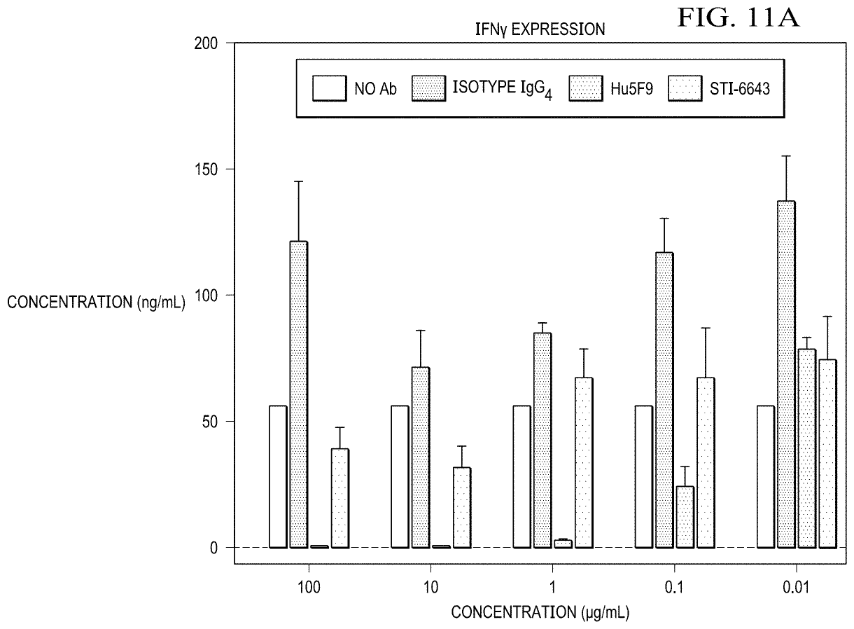

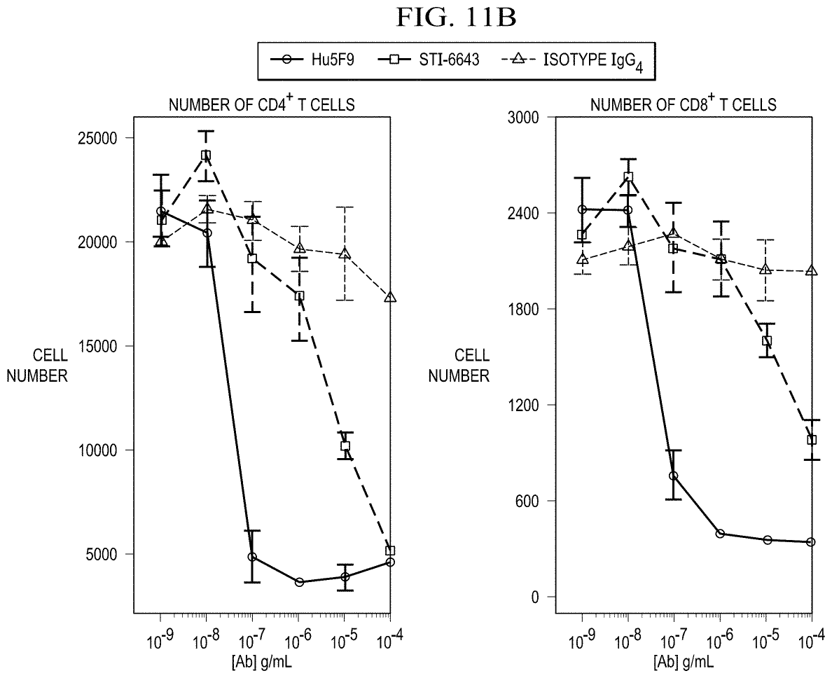

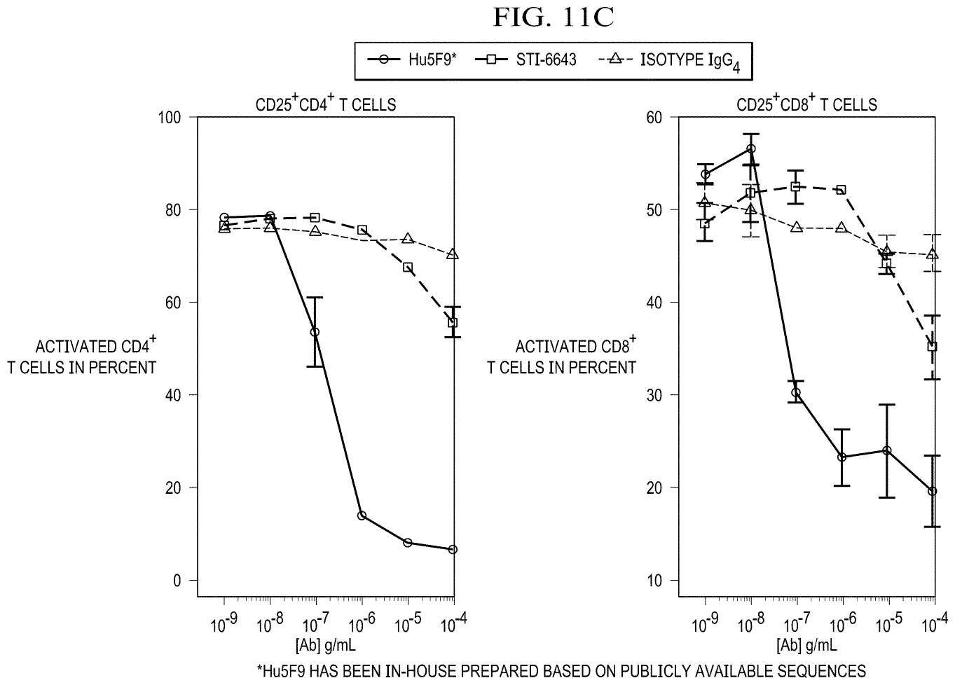

On day 3, the cells were spun down for 420 g for 5 minutes. Supernatants were transferred to a new 96-well plate and the IFNγ cytokine content was measured using the proinflammatory panel 1 (human) kit from Meso Scale Discovery (MSD; Cat. No. K15049D) by following the manufacturer recommendations (FIG. 11A). In FIG. 11A, each concentration along the x-axis includes from left to right: no antibody control; isotype IgG4 control; Hu5F9; and STI-6643. Total number of CD4⁺, CD8⁺, CD19⁺ and CD56⁺ cells (FIG. 11B) as well as percentage of activated CD4⁺CD25⁺ and CD8⁺CD25⁺ T cells (FIG. 11C) were evaluated as follows: Cells were washed using cold FACS buffer (Dulbecco's Phosphate Buffered Saline supplemented with 2 mM EDTA and 2% Fetal Bovine Serum). Then, cells were incubated for 20 minutes at 4° C. with an antibody mixture composed of PE-conjugated CD8 (clone SK1; Biolegend, Cat. no. 344706, lot# B267519), V421-conjugated CD4 (clone OKT4; Biolegend, Cat. no. 317434, lot# B280597), AF647-conjugated CD25 (clone M-A251; Biolegend, Cat. no. 356128, lot# B269090). After washing cells twice with 150 μL/well of FACS buffer, the cells were resuspended in 200 μL/well of FACS buffer and acquired immediately by flow cytometry and analyzed using the FlowJo software. Data represent the mean +/−S.E.M of triplicate values for each point.

Conclusion: STI-6643 sustains activated T cell viability and IFNγ response in a pro-inflammatory environment.

### Example 12: Dose-Efficacy Study of STI-6643 in the RAJI Tumor Model

Fox Chase SCID mice were inoculated intravenously with luciferase expressing Raji cells (RAJI-Fluc) and randomized into different treatment groups (30 mpk STI-6643, n=16 mice; 10 mpk STI-6643, n=24 mice; 1 mpk STI-6643, n=24 mice; 0.1 mpk STI-6643, n=16 mice; 10 mpk isotype control, n=24 mice and 30 mpk isotype control , n=8 mice).

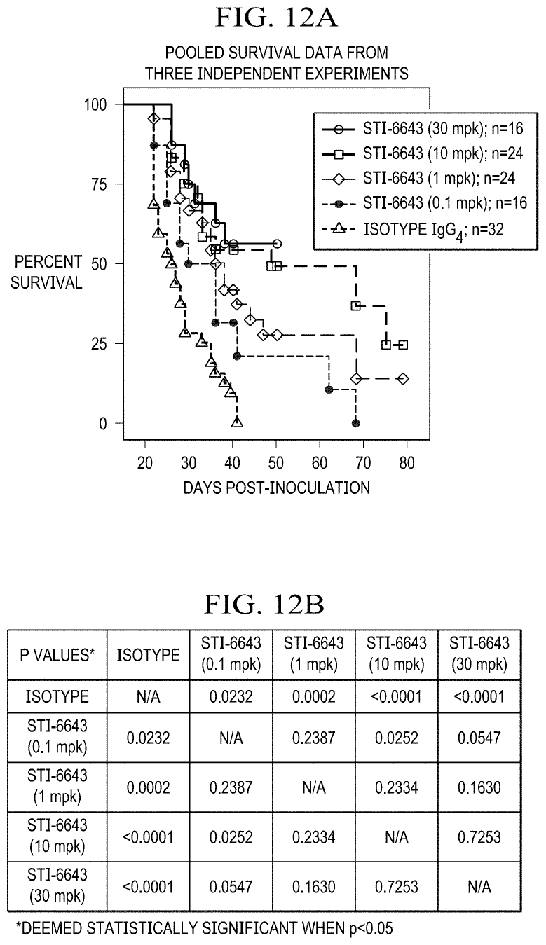

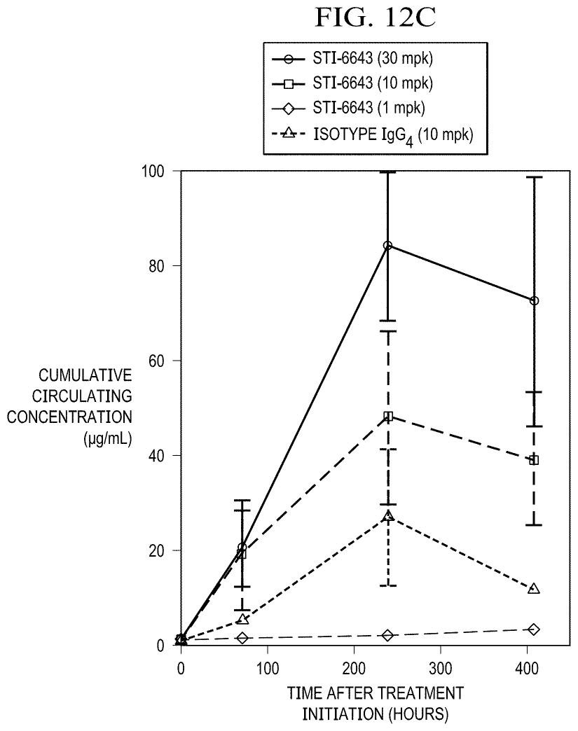

Treatments (0.1-1-10 or 30 mpk of STI-6643, and 10 or 30 mpk for human IgG4 of isotype control) were given thrice a week for 2 to 3 consecutive weeks (total 6 to 8 doses) starting on day 7 post RAJI-Fluc inoculation. Kaplan-Meier survival analysis was performed using the GraphPad Prism software by combining the data from three independent experiments, each containing both STI-6643 and isotype treated groups. Time of survival was determined for each animal as the first day where signs of hindlimb paralysis were observed. The percent survival results are shown in FIG. 12A. p values comparing each treatment group with the others are shown in the Table (p<0.05) is considered statistically significant) (see the Table in FIG. 12B).

Circulating antibody concentrations were evaluated in treated animals. Blood samples were collected as follows: For each sample 10 4 of whole blood was mixed with 90 μL of Blocker™ Casein in PBS (Thermo Fisher; Cat. 37528) and quickly stored at −80° C. until the ELISA was run. A multi-array 96-well plate (Meso Scale Discovery, Cat. L15XA-3) was coated with unlabeled mouse anti-human IgG (CH2 domain) antibody (Thermo Fisher; Cat. MA5-16929, lot. UE2781631A) at 2 μg/mL in 1× PBS (50 μL/well). After washing with 1× KPL washing solution (Sera care; Cat. 5150-0008, lot. 10214473), the plate was blocked with Blocker™ Casein in PBS for 1 hour at 37° C. A standard curve was prepared using STI-6643 mAb in Blocker™ Casein in PBS by performing serial dilutions covering concentrations ranging from 50 to 0 ng/mL. Subsequently, the 96-well plate was washed and both blood samples (diluted 1:10,000) and standard curve samples were transferred into the wells (50 μL/well) and incubated for 2 hours at room temperature (RT) under slow shaking. Plate was washed thrice with 1× KPL washing solution and incubated for 1 hour at RT with a goat anti-human/NHP SULFO-TAG antibody from Meso Scale Discovery (Cat. D20JL-6, lot. W00180455) at a 1:500 dilution in Blocker™ Casein in PBS (25 μL/well). After washing thrice with 1× KPL washing solution, the presence of antibodies was revealed by adding 150 μL/well of 2× Read buffer (Meso Scale Discovery; Cat. R92TC-3, lot. Y0140368). The plate was immediately read on an MSD instrument (Meso Sector S600 Model 1201; Serial number 1201160919484). Data are given as a mean +/−S.D. The results are shown in FIG. 12C.

Conclusion: STI-6643 showed dose-dependent anti-tumor activity in the RAJI tumor model.

### Example 13: Efficacy Study in the NCI-H82 Lung Solid Tumor Model

Balb/SCID mice were inoculated subcutaneously into the right flank with 5.0E+06 NCI-H82 lung tumor cells prepared in 1× HBSS (150 μL/mouse and randomized into different treatment groups on day 12 (when a tumor bump was present in more than 80% of the animals). If a mouse did not present a tumor bump at treatment start, it was removed from the study. STI-6643 (n=9 mice) or isotype control (n=8 mice) human IgG4 antibodies were administered systemically at 90 mg/kg by subcutaneous injections (150 μL/mouse) every other day for a total of 6 doses.

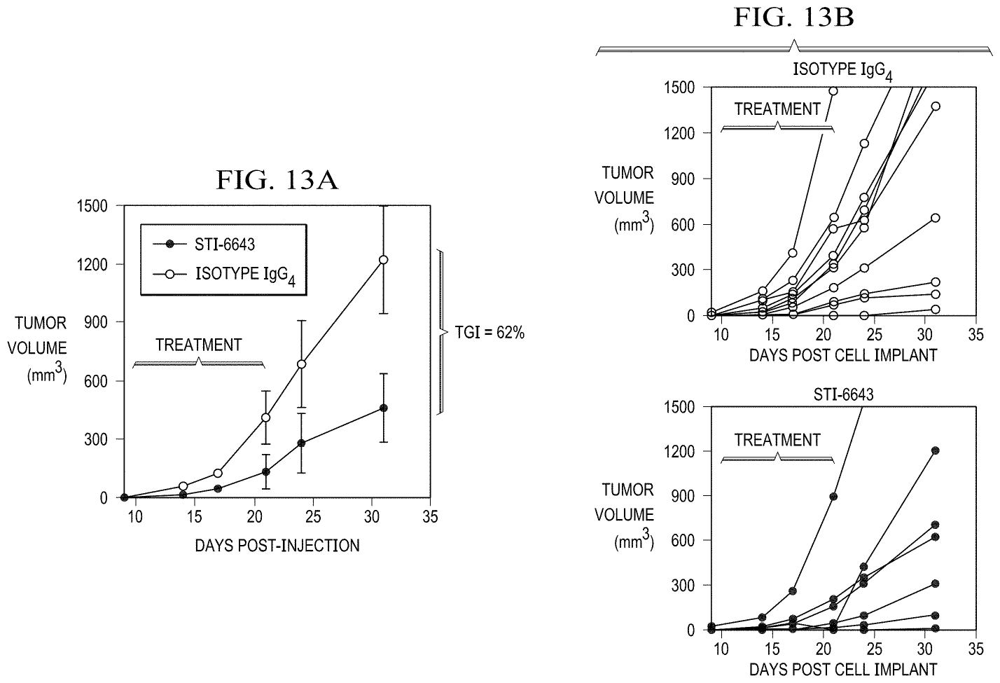

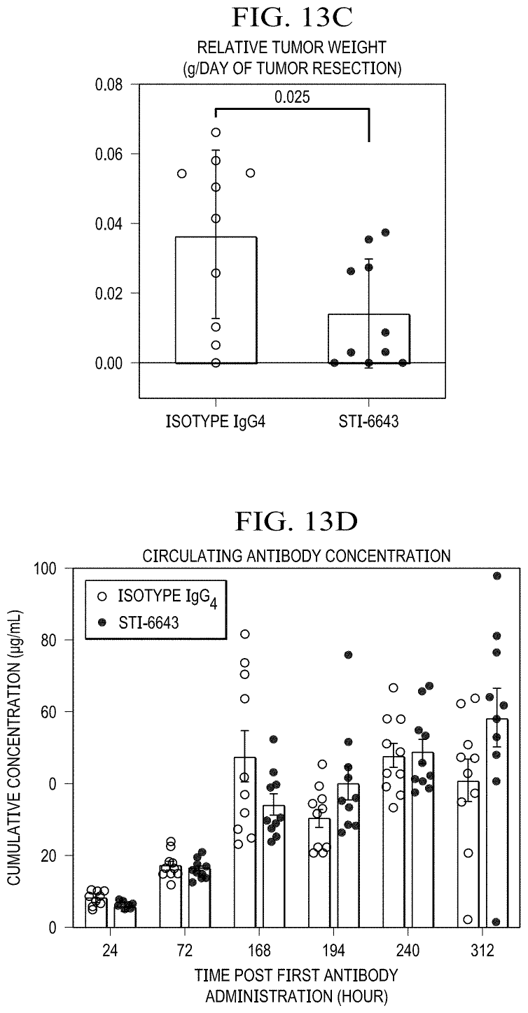

Average (FIG. 13A) and individual (FIG. 13B) tumor volumes were measured over time and tumor growth inhibition (TGI) calculated at the end of the study day 31 post tumor cell implantation). Tumors were resected and weighted at the time of take down (FIG. 13C). Data are displayed as an average of relative tumors weights (tumor weight/day of tumor resection). Average data represents the mean +/−S.E.M (p<0.05 is considered statistically significant). Circulating antibody concentrations were evaluated in treated animals (FIG. 13D). in FIG. 13D, each time post along the x-axis includes from left to right: isotype IgG4 control; and STI-6643. Blood samples were collected as follows: For each sample 10 μL of whole blood was mixed with 90 μL of Blocker™ Casein in PBS (Thermo Fisher; Cat. 37528) and quickly stored at −80° C. until the ELISA was run. A multi-array 96-well plate (Meso Scale Discovery, Cat. L15XA-3) was coated with unlabeled mouse anti-human IgG (CH2 domain) antibody (Thermo Fisher; Cat. MA5-16929, lot. UE2781631A) at 2 μg/mL in 1× PBS (50 μL/well). After washing with 1× KPL washing solution (Sera care; Cat. 5150-0008, lot. 10214473), the plate was blocked with Blocker™ Casein in PBS for 1 hour at 37° C. A standard curve was prepared using STI-6643 mAb in Blocker™ Casein in PBS by performing serial dilutions covering concentrations ranging from 50 to 0 ng/mL. Subsequently, the 96-well plate was washed and both blood samples (diluted 1:10,000) and standard curve samples were transferred into the wells (50 μL/well) and incubated for 2 hours at room temperature (RT) under slow shaking. Plate was washed thrice with 1× KPL washing solution and incubated for 1 hour at RT with a goat anti-human/NHP SULFO-TAG antibody from Meso Scale Discovery (Cat. D20M-6, lot. W00180455) at a 1:500 dilution in Blocker™ Casein in PBS (25 μL/well). After washing three times with 1× KPL washing solution, the presence of antibodies was revealed by adding 150 μL/well of 2× Read buffer (Meso Scale Discovery; Cat. R92TC-3, lot. Y0140368). The plate was immediately read on an MSD instrument (Meso Sector 5600 Model 1201; Serial number 1201160919484). Data are given as a mean +/−S.D.

Conclusion: STI-6643 showed anti-tumor activity in the NCI-H82 tumor model.

### Example 14: Dose Efficacy Study in the NCI-H82 Lung Solid Tumor Model

SCID mice were inoculated subcutaneously into the right flank with 5.0E+06 NCI-H82 lung tumor cells prepared in HBSS 1× (150 μL/mouse and randomized into different treatment groups when a tumor bump was present in more than 80% of the animals (on day 11 or 12). If a mouse did not present a tumor bump at treatment start, it was removed from the study.

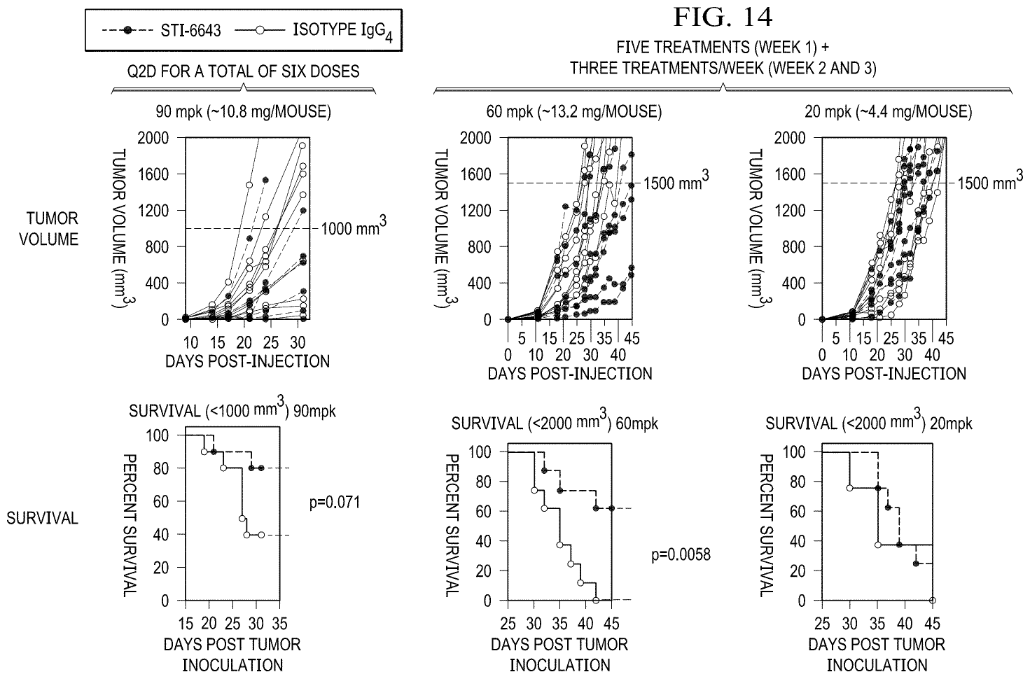

STI-6643 or isotype control human IgG4 antibodies were administered systemically at 20, 60 or 90 mpk (mg/kg) by subcutaneous injections (150 μL/mouse) using a different treatment schedule. Treatments for the 20 and 60 mpk groups were 5 consecutive injections on week 1 then 3 times per week for weeks 2 and 3. For the 90 mpk group, the treatment schedule was every other day (Q2D) for a total of 6 doses. Individual tumor volumes and Kaplan-Meier survival curves were obtained for each concentration tested (FIG. 14). The upper curve in each Kaplan-Meier plot is based on STI-6643 treated mice, and the lower curve is based on Isotype treated mice. Survival was calculated based on a tumor volume of 1,500 mm³ for 20 and 60 mpk groups and 1,000 mm³ for the 90 mpk group (as this study was terminated earlier on day 31). p<0.05 is considered statistically significant. The tumor volume and percent survival results are shown in FIG. 14.

Conclusion: STI-6643 showed dose-dependent anti-tumor activity in the NCI-H82 tumor model, when comparing the total amount of antibody injected in mg/mouse.

### Example 15: Efficacy Study in the A375 Melanoma Solid Tumor Model

NBSGW mice were inoculated subcutaneously into the right flank with 5.0E+06 A375 lung tumor cells prepared in HBSS 1× (150 μL/mouse and randomized into different treatment groups on day 7 (when a tumor bump was present in more than 80% of the animals). If a mouse did not present a tumor bump at treatment start, it was removed from the study.

STI-6643 (n=10 mice) or isotype control (n=10 mice) human IgG4 antibodies were administered systemically at 20 mg/kg by subcutaneous injections (150 μL/mouse) every other day for a total of 6 doses.

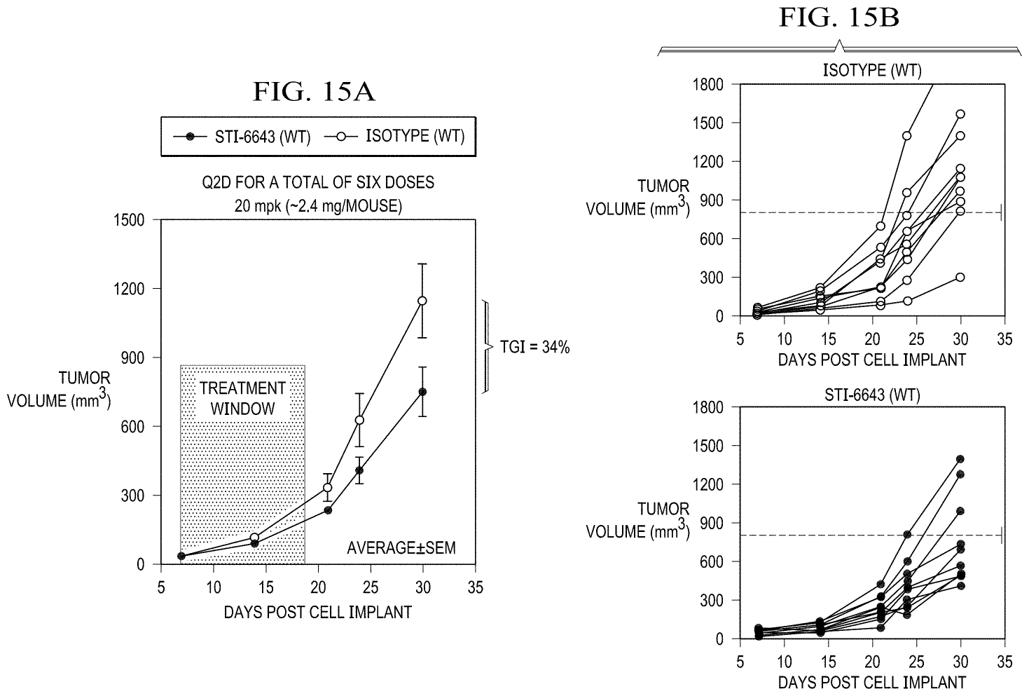

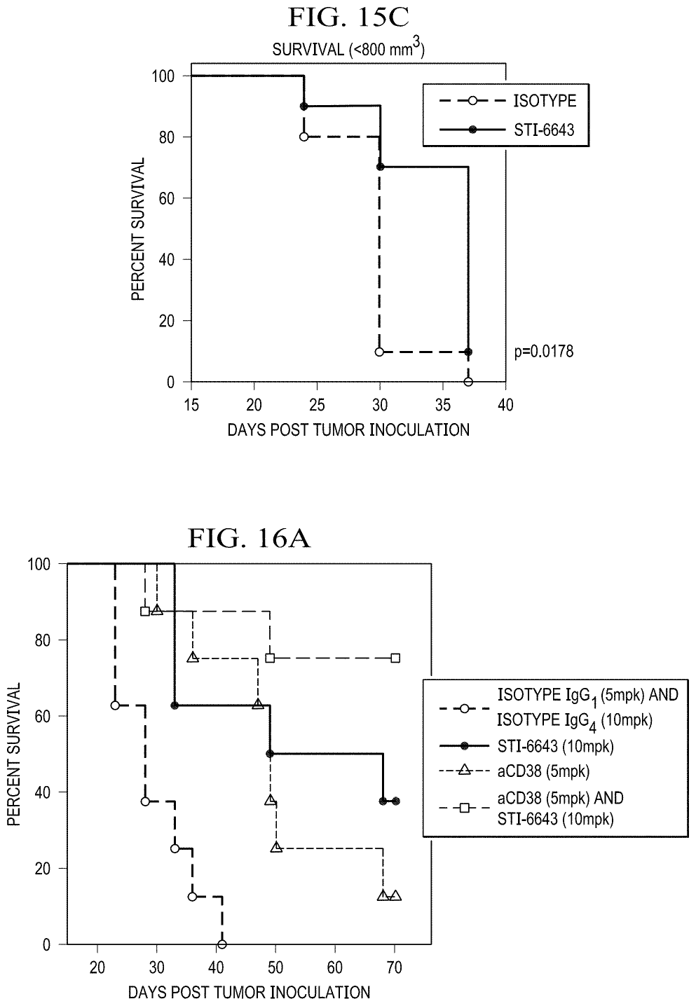

Average (FIG. 15A) and individual (FIG. 15B) tumor volumes were measured over time and tumor growth inhibition (TGI) calculated at the end of the study day 31 post tumor cell implantation). Kaplan-Meier survival curves were plotted using the GraphPad Prism software (FIG. 15C). Survival was calculated based on a tumor volume of 800 mm³. p<0.05 is considered statistically significant.

Conclusion: STI-6643 showed anti-tumor activity at 20 mpk in the A375 tumor model.

### Example 16: Efficacy Study of Anti-CD47 (STI-6643) in Combination with Daratumumab (Anti-CD38)

Fox Chase SCID mice (n=8 per group) transplanted intravenously with luciferase expressing Raji cells (RAJI-Fluc) were treated with either 5 mpk (mg/kg) of anti-CD38 Daratumumab alone, 10 mpk of STI-6643 alone, a combination of (5 mpk Daratumumab+10 mpk of STI-6643) or a combination of (5 mpk human IgG₁+10 mpk of human IgG₄ isotype controls). Treatments were given on day 7, 9, 11, 14, 16 and 18 post RAJI-Fluc inoculation.

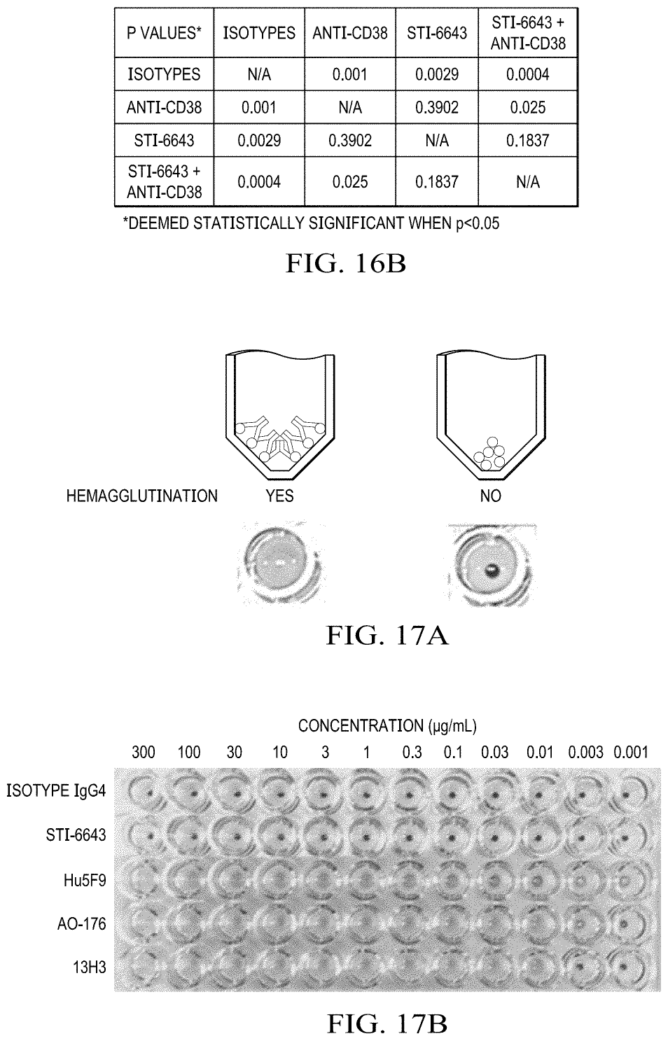

Kaplan-Meier survival analysis was performed using the GraphPad Prism software. Time of survival was determined for each animal as the first day where signs of hindlimb paralysis were observed. The percent survival graph is shown in FIG. 16A. p values comparing each treatment group with the others are shown in the Table (p<0.05 is considered statistically significant) (see the Table in FIG. 16B).

Conclusion: Combination Therapy with Anti-CD47 Antibody and Daratumumab Prolonged Survival in Disseminated Human RAJI xenograft Mouse Models.

### Example 17. Anti-CD47 antibody STI-6643 Drastically Reduces Hemagglutination as Compared to Anti-CD47 Antibodies Hu5F9, A0176, and 13H3

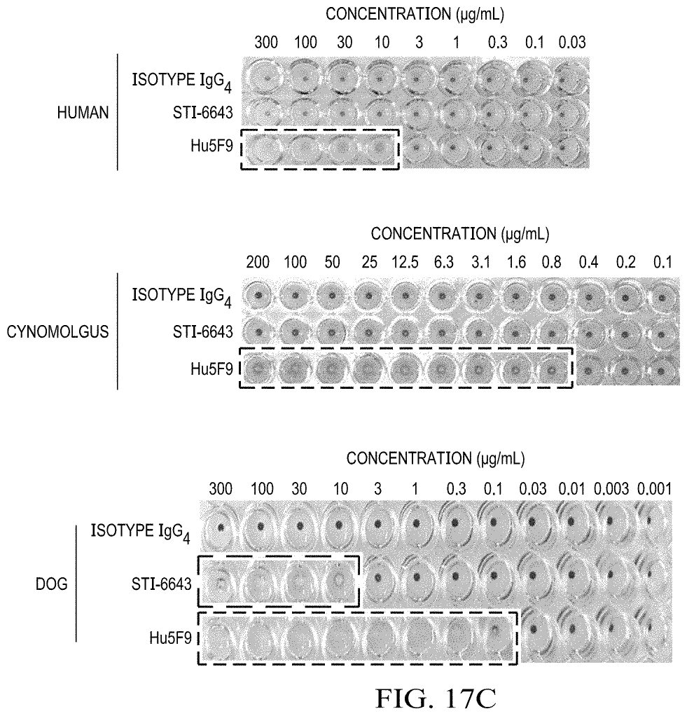

Red Blood Cells (RBCs) preparation: RBCs were prepared from 4 mL of peripheral blood from a healthy human donor, cynomolgus monkey, and beagle dog using the methods of Example 9. For the hemagglutination assay, 50 μL of 0.5% RBCs of human, monkey, and dog origin were added to each well of a U-bottom 96-well plate and the plate was maintained at RT. Antibodies used in this assay were: anti-CD47 IgG₄ STI-6643; anti-CD47 IgG4 Hu5F9 (Liu et al. (2015) PLoSONE, incorporated herein by reference); anti-CD47 IgG₄ AO-176 (Puro et al. (2020) Mol. Cancer Ther. 19:835-846, incorporated herein by reference), and anti-CD47 IgG₄ 13H3 (see US2020/0140565A1, incorporated herein by reference), and isotype human IgG₄ control. Serial dilutions of antibodies were made in an ultra-low attachment 96-well plate in 1× PBS (2-fold dilutions). The antibody dilutions were transferred into the plate containing RBCs (100 μL/well, starting antibody concentration: 300 μg/mL) and slowly mixed a few times with a multichannel pipet. The plate was placed in a tissue culture incubator (5% CO₂, 37° C.) for a ˜20 h incubation. Negative results (no hemagglutination) appeared as dots in the centre of round-bottomed plates. Positive results (hemagglutination) formed a uniform reddish color across the well. FIG. 17A (top) provides an example of positive and negative results. FIG. 17B (bottom) shows the results of hemagglutination assays with anti-CD47 antibodies STI-6643, Hu5F9, AO-176, and 13H3, demonstrating that unlike the other anti-CD47 antibodies tested, STI-6643 does not induce hemagglutination even at high concentration.

FIG. 17C demonstrates that the STI-6643 antibody does not induce hemagglutination of human and cynomolgus (monkey) RBCs, although some hemagglutination is seen to occur with dog RBCs at concentrations of antibody greater than 3 μg/mL.

### Example 18. Binding of anti-CD47 Antibodies STI-6643and Hu5F9 to Human, cynomologus, and Canine RBCs

RBCs of were prepared as described in Example 17, above. Binding to RBCs was tested in a multiwell format. FACS Buffer (1× PBS+2% FCS+2 mM EDTA) was used throughout the assay. 1.25E+06 RBCs per well were plated in a V-bottom 96-well plate in 50 μL 1× PBS. 50 μL of FACS buffer at RT containing various concentrations of anti-CD47 IgG₄ antibodies (STI-6643 or Hu5F9) or isotype IgG₄ control were added. Cells were incubated in the presence of antibodies for 45 min at 37° C. and gently mixed with a multichannel pipet every 15 min. Then, cells were washed twice with 100 μL/well of FACS buffer at RT, spun down by centrifugation (524×g for 3 min) and supernatants were aspirated. The RBC pellets were resuspended in 50 μL/well of FACS buffer at RT containing APC-labelled anti-human IgG Fc antibody (BioLegend; clone HP6017, Cat. No. 409306; Lot. B86581) diluted at 1:200 and incubated for 30 min at 37° C. Cells were washed twice with 150 μL/well of FACS buffer at RT, spun down by centrifugation (524 g; 3 min) and supernatants were aspirated and discarded. Cells were then fixed by resuspending the pellets in 100 μL of fixation buffer (BioLegend; Cat. No. 420801) for a 20 min incubation at 4 ° C. After addition of 100 μL/well of 4° C-cold FACS buffer, cells were spun down by centrifugation (524 g; 3 min), the supernatants were removed by slow aspiration and the pellets resuspended in 200 μL of 4° C-cold FACS Buffer. Samples were analyzed by flow cytometry within 24 hours.

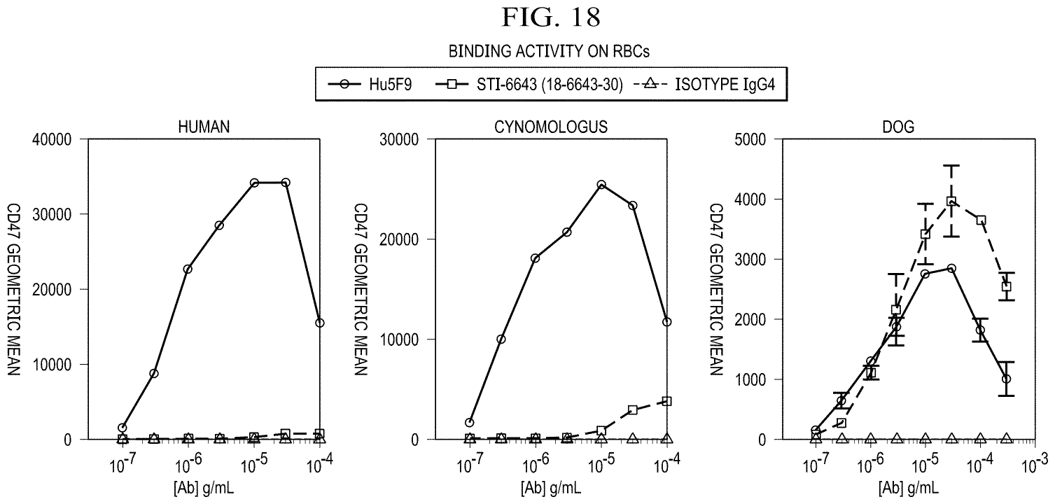

FIG. 18 provides the results of the anti-CD47 binding assays using RBCs of human, monkey, and dog. In the graphs showing binding to human and cynomologus RBCs, the upper curve in the graph shows binding by anti-CD47 antibody Hu5F9, which shows binding increasing as the antibody concentration rises above 10⁻⁷ g/ml. STI-6643 shows no specific binding of human RBCs in this assay (the curve coincides with the isotype control) and binding of cynomologus RBCs occurs to a much lesser degree at concentrations above about 10⁻⁵ g/ml. The upper curve of the rightmost graph, showing binding to dog RBCs, is binding by the STI-6643 antibody and below it is the curve of Hu5F9 binding to dog RBCs. Both of these antibodies bind dog RBCs at concentrations above about 10⁻⁷ g/ml.

### Example 19. Binding of anti-CD47 Antibodies STI-6643, Hu5F9, 13H3, and AO-176 to Raji tumor Cells and Human RBCs

RBCs were prepared according to Example 17, above, and FACS Buffer (1× PBS+2% FCS+2 mM EDTA) was used throughout the assays.

30,000 RAJI cells per well were plated in a V-bottom 96-well plate. Plates were spun down by centrifugation at 524 g for 3 min and the supernatant was removed by quickly flicking the plate. Cells were resuspended in 50 μL/well of FACS buffer at RT containing various concentrations of anti-CD47 IgG₄ antibodies (STI-6643, Hu5F9, AO-176 and 13H3) or isotype IgG₄ control (1:10 serial dilutions starting at 100 μg/mL) and incubated for 25 min at 37° C. Cells were then washed with 150 μL/well of FACS buffer at RT, spun down by centrifugation (524 g; 3 min) and supernatants were removed by quickly flicking the plate. Cells were resuspended in 50 μL/well of FACS buffer at RT containing APC-labelled anti-human IgG Fc antibody (BioLegend; clone HP6017, Cat. No. 409306; Lot. B86581) diluted at 1:200 and incubated for 20 min at 37° C. Cells were washed with 150 μL/well of FACS buffer at RT, spun down by centrifugation (524 g; 3 min) and supernatants were removed by quickly flicking the plate. The cells were fixed by resuspending the pellets in 100 μL of fixation buffer (BioLegend; Cat. No. 420801) for a 20-min incubation at 4° C. After addition of 4° C-cold FACS buffer (100 μL/well) the samples were analysed by flow cytometry within 24 hours.

RBCs (1.25E+06 per well) were plated in a V-bottom 96-well plate in 50 μL 1× PBS. 50 μL of FACS buffer at RT containing various concentrations of anti-CD47 IgG₄ antibodies (STI-6643, Hu5F9, 13H3 and AO-176) or isotype IgG₄ control were added. Cells were incubated in the presence of antibodies for 45 min at 37° C. and gently mixed with a multichannel pipet every 15 min. Then, cells were washed twice with 100 μL/well of FACS buffer at RT, spun down by centrifugation (524 g; 3 min) and supernatants were aspirated. The RBC pellets were resuspended in 50 μL/well of FACS buffer at RT containing APC-labelled anti-human IgG Fc antibody (BioLegend; clone HP6017, Cat. No. 409306; Lot. B86581) diluted at 1:200 and incubated for 30 min at 37 ° C. Cells were washed twice with 150 μL/well of FACS buffer at RT, spun down by centrifugation (524 g; 3 min) and supernatants were aspirated and discarded. Then, cells were fixed by resuspending the pellets in 100 μL of fixation buffer (BioLegend; Cat. No. 420801) for a 20 min incubation at 4° C. After addition of 100 μL/well of 4° C-cold FACS buffer, cells were spun down by centrifugation (524 g; 3 min), the supernatants were removed by slow aspiration and the pellets resuspended in 200 μL of 4° C-cold FACS Buffer. Samples were analysed by flow cytometry within 24 hours.

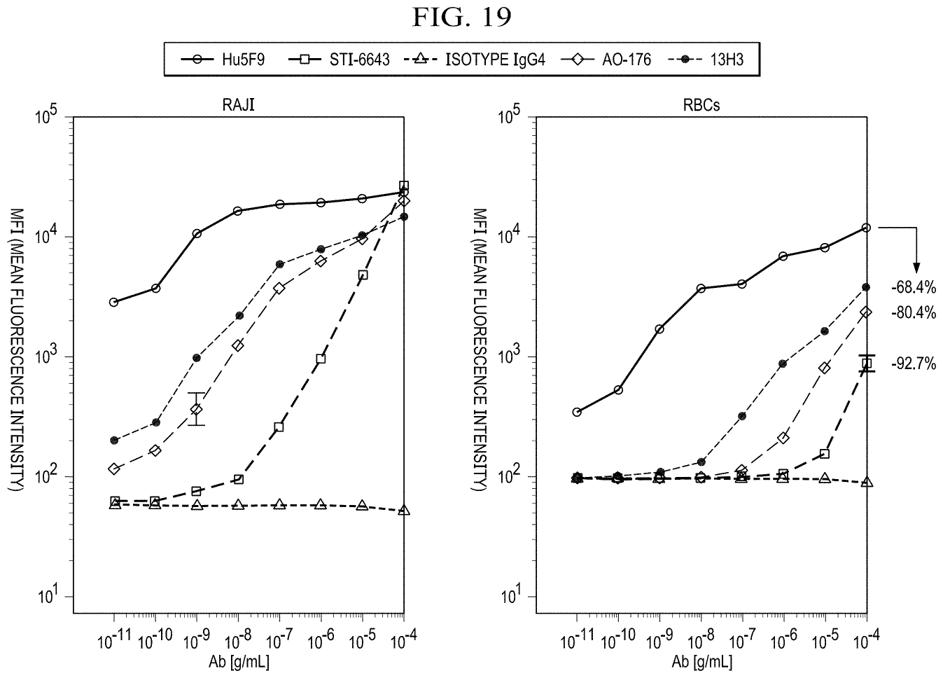

FIG. 19 shows binding to Raji cells by the anti-CD47 antibodies in the graph at the left of the figure. The upper curve is antibody Hu5F9, followed by antibodies 13H3 and AO-176 showing very similar binding curves, and then antibody ST-6643. The isotype control is a flat line at low MFI (no concentration dependence). The graph on the right of the figure shows that antibody Hu5F9 has the highest level of RBC binding at all concentrations, followed by the 13H3 antibody (reduced by approximately 68% at the maximal binding concentration) with respect to Hu5F9 binding) and then the AO-176 binding curve (reduced by approximately 80% with respect to Hu5F9 binding at the maximal binding concentration). STI-6643 is the lowest curve, showing the lowest amount of binding to RBCs at concentrations less than about 10⁻⁷ g/ml, reduced by approximately 93% with respect to the binding of RBCs by anti-CD47 antibody Hu5F9 at the maximal binding concentration.

### Example 20. Assessment of Immune Cells after PBMC incubation with anti-CD47 Antibodies STI-6643, Hu5F9, 13H3, and AO-176

To test the effects of anti-CD47 antibody STI-6643 on normal immune cells, assays were performed where PBMCs were incubated with anti-CD47 antibodies and then stained for markers of immune cell types.

Freshly purified PBMCs from three different donors were mixed in equivalent proportions and plated at 0.5E+06 cells/well in 200 μL of RPMI1640 10% human AB serum (Valley Biomedical, Cat. No. HP1022, Lot. 6F1131)+P/S at 37° C. in a flat-bottom 96-well plate in the presence of isotype IgG4 Ab, anti-CD47 mAbs STI-6643, Hu5F9, AO-176 or 13H3 at final concentrations ranging from 1 ng/mL to 100 μg/mL. After a 6-day incubation at 37° C., cells were spun down by centrifugation for 3 min at 524 g, supernatant was discarded by flicking the plate, cells were washed with 200 μL FACS buffer at 4° C. and stained for 30 minutes at 4° C. with the following fluorochrome-conjugated mAbs diluted in 100 μL of FACS buffer at 4° C.: anti-human CD4-PE (clone OKT4, BioLegend, Cat. No. 317410, Lot. B264363, 2 μL/well), anti-human CD8-FITC (clone HIT8a, BioLegend, Cat. No. 300906, Lot. B275277, 2μL/well), anti-human CD19-APC-Cy7 (clone SJ25C1, BioLegend, Cat. No. 363010, Lot. B276795, 2 μL/well), and CD56-PB (clone HCD56, BioLegend, Cat. No. 318326, Lot. B280451, 2 μL/well). Cells were washed by adding 100 μL of FACS buffer at 4° C., centrifugation for 3 min at 524 g and removing supernatant by flicking the plate, then resuspended in 100 μL of fixation buffer (BioLegend; Cat. No. 420801) for 20 minutes minimum at 4° C. 100 μL of FACS buffer at 4° C. was added without washing and the numbers of CD4⁺, CD8⁺, CD19⁺ and CD56⁺ cells recovered at the end of the 6-day incubation period are analysed by flow cytometry, data are presented as a mean +/−S.E.M.

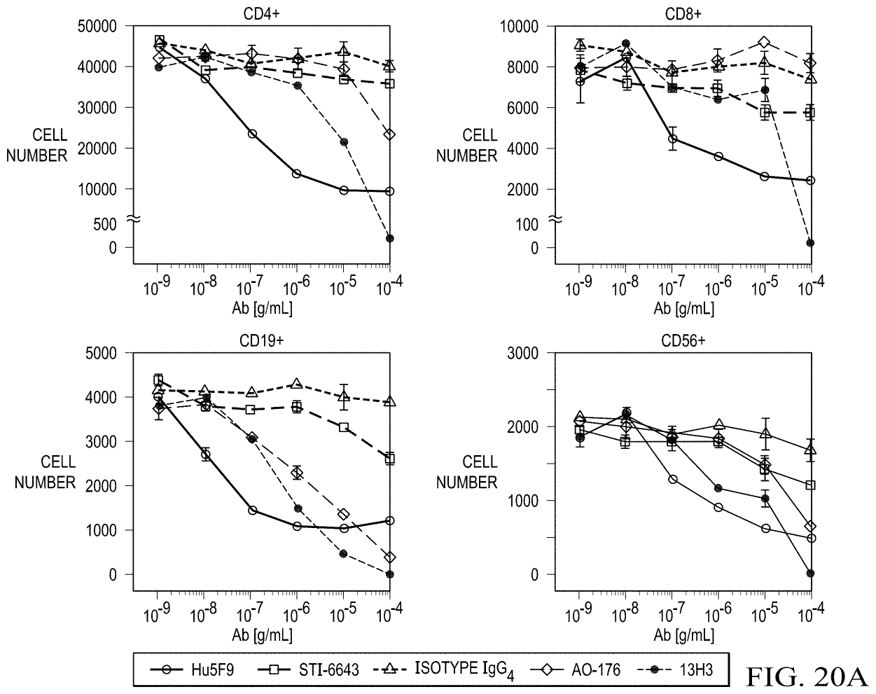

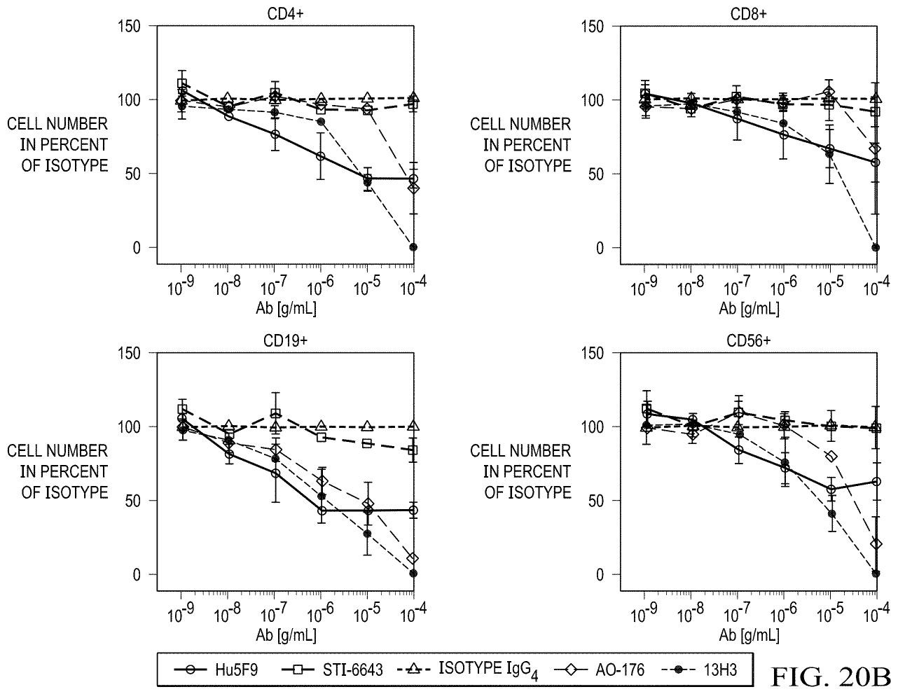

One representative experiment is shown in FIG. 20A, where data are presented as a mean +/−S.E.M. of the numbers of cells recovered after incubation with the anti-CD47 antibodies. In FIG. 20B, data are presented as the percentage of the number of cells recovered in presence of Isotype IgG4 at the same concentration. Data from 2 experiments (13H3 and AO-176) or 4 experiments (Isotype IgG4, STI-6643 and Hu5F9) were used to generate the normalized graphs. Data are presented as a mean +/−S.E.M. of 2-4 independent experiments.

In the graphs of A), the greatest reductions in cell numbers for CD4+, CD8+, CD19+, and CD56+ cells are seen for antibody Hu5F9 and 13H3, with the AO-176 antibody also resulting in a concentration-dependent reduction in CD19+ cells. In the graphs of B), based on percentages of cells recovered after isotype antibody incubation, the percentage of recovered cells after incubation with STI-6643 tracks the isotype control at the top of the graph, showing concentration-dependent loss of cells only to a slight degree at the highest concentration of antibody in the case of CD19+ cells.

### Example 21: Efficacy Study of Anti-CD47 (STI-6643) in a MDA-MB-231 Pseudo-Humanized Mouse Model

On day 0, NSG-Tg(Hu-IL15) mice (stock number 30890; The Jackson Laboratory) were humanized using an intraperitoneal injection of 1.0E+07 human peripheral blood mononuclear cell (PBMCs). On day 8, mice were inoculated subcutaneously into the right flank with 5.0E+06 MDA-MB-231 breast tumor cells prepared in HBSS 1× (100 μL/mouse) and randomized into different treatment groups on day 15 (when tumor size reached 50-100 mm³ in more than 90% of the animals). If a mouse did not present a tumor bump at treatment start, it was removed from the study. STI-6643 antibody was administered systemically at 0.1, 1 and 10 mg/kg by subcutaneous injections (100 μL/mouse; n=5 mice/group) every other day for a total of 3 doses. Individual and average tumor volumes were measured over time. Data were plotted and statistic obtained using the GraphPad Prism software. Statistical analyses were performed using the 2-way ANOVA, Sidak multiple comparison test. p<0.05 is considered statistically significant.

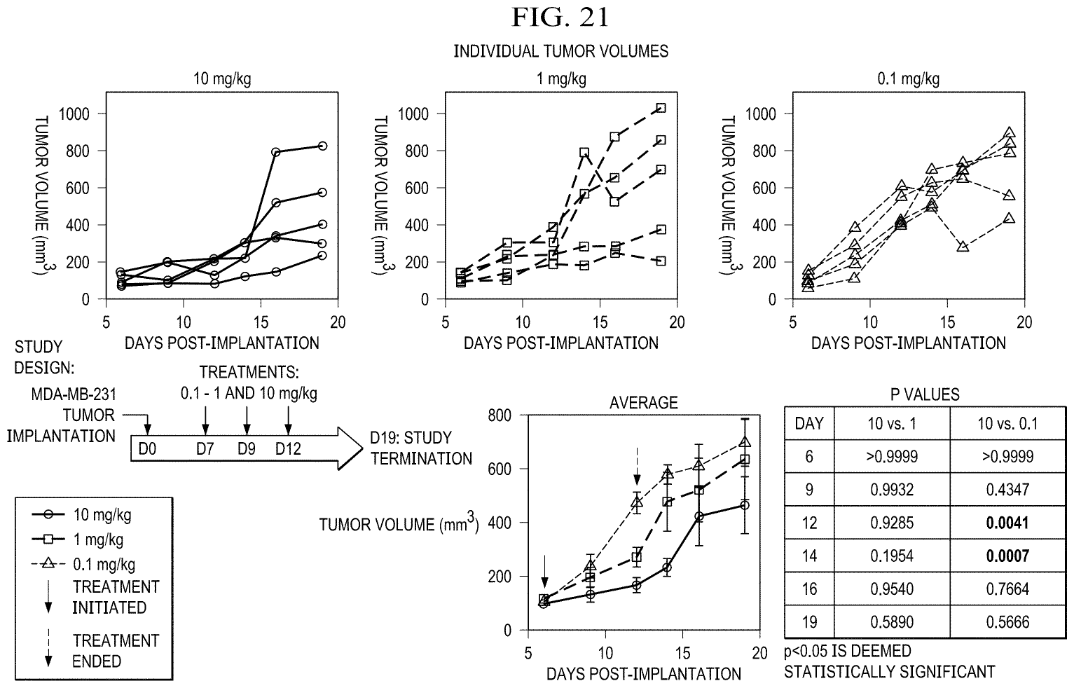

FIG. 21 provides the results depicting that treatment with STI-6643 resulted in reduced tumor growth in a humanized MDA-MB-231 tumor model.

1. A composition comprising: (i) a first antibody or an antigen binding fragment thereof that binds an epitope of a CD47 antigen, and (ii) a second antibody that binds an epitope of a CD20 antigen or a CD38 antigen, wherein the first antibody binds to CD47 antigen and blocks binding between CD47 antigen and SIRPα antigen.

2. A method for killing at least one cancer cell in a population of cancer cells, wherein the at least one cancer cell overexpresses CD47 antigen, the method comprising:
contacting the at least one cancer cell with a therapeutically effective amount of a first antibody or an antigen binding fragment thereof that binds CD47 antigen and a second antibody that binds CD20 antigen or binds CD38 antigen, wherein the first antibody binds to CD47 antigen and blocks binding between CD47 antigen and SIRPα antigen, and wherein the second antibody comprises Fc portion that binds an Fcγ receptor on an effector cell.

3. A method for treating a subject having a cancer that overexpresses CD47 antigen, the method comprising: administering to the subject a therapeutically effective amount of a first antibody or an antigen binding fragment thereof that binds CD47 antigen and a second antibody that binds CD20 antigen or CD38 antigen, wherein the first antibody binds to CD47 antigen and blocks binding between CD47 antigen and SIRPα antigen, and wherein the second antibody comprises Fc portion that binds an Fcγ receptor on an effector cell.

4. The composition of claim 1, wherein the antigen binding fragment of the first antibody comprises a Fab fragment, F(Ab′)2 fragment or scFv fragment.

5. The composition of claim 1, wherein the second antibody comprises an Fc portion that binds an Fcγ receptor on an effector cell.

6. The composition of claim 1, further comprising a pharmaceutically acceptable excipient.

7. The composition of claim 1, wherein the first antibody comprises an IgG4 type anti-CD47 antibody.

8. The composition of claim 1, wherein the first antibody comprises a fully human anti-CD47 antibody.

9. The composition of claim 1, wherein the first antibody comprises a variable heavy chain domain comprising the amino acid sequence of SEQ ID NO:1 and a variable light chain domain comprising the amino acid sequence of SEQ ID NO:2. (STI-6643)

10. The composition of claim 1, wherein the CD47 antigen comprises a human CD47 antigen comprising the amino acid sequence of SEQ ID NO:5 or a portion thereof.

11. The composition of claim 1, wherein the first antibody exhibits reduced hemagglutination when contacted with human red blood cells, compared to anti-CD47 antibody (Hu5F9), wherein the Hu5F9 antibody comprises a variable heavy chain domain comprising amino acids 1-117 of the amino acid sequence of SEQ ID NO:3 and a variable light chain domain comprising amino acids 1-112 of the amino acid sequence of SEQ ID NO:4.

12. The composition of claim 1, wherein the first antibody mediates phagocytosis killing of a cell expressing CD47 antigen when contacted with human macrophage cells (e.g., CD14+ macrophage cells).

13. The composition of claim 1, wherein the second antibody comprises an IgG1 type anti-CD20 antibody or an IgG1 type anti-CD38 antibody.

14. The composition of claim 1, wherein the second antibody induces antibody dependent cell-mediated cytotoxicity (ADCC) in the presence of effector cells.

15. The composition of claim 1, wherein the second antibody comprises
a) a chimeric anti-CD20 antibody (e.g., Rituximab); a humanized anti-CD20 antibody (e.g., Obinutuzumab); or a fully human anti-CD20 antibody (e.g., Ofatumumab); or
b) an anti-CD38 antibody (e.g., Daratumumab, FIG. 18B); or any one anti-CD38 antibody listed in Tables A, B or C listed in FIGS. 18C-D.

16. The composition of claim 1, wherein the second antibody comprises
a) a variable heavy chain domain comprising amino acids 1-121 of the amino acid sequence of SEQ ID NO:6 and a variable light chain domain comprising amino acids 1-106 of the amino acid sequence of SEQ ID NO:7 (Rituximab);
b) a variable heavy chain domain comprising amino acids 1-119 of the amino acid sequence of SEQ ID NO:8 and a variable light chain domain comprising amino acids 1-115 of the amino acid sequence of SEQ ID NO:9 (Obinutuzumab);
c) a variable heavy chain domain comprising amino acids 1-122 of the amino acid sequence of SEQ ID NO:10 and a variable light chain domain comprising amino acids 1-107 of the amino acid sequence of SEQ ID NO:11 (Ofatumumab); or
d) a variable heavy chain domain comprising the amino acid sequence of SEQ ID NO:16 and a variable light chain domain comprising the amino acid sequence of SEQ ID NO:17 (Daratumumab); or
e) any one of the paired variable heavy chain domain and variable light chain domain listed in Tables A, B, and C (FIGS. 18C-E, respectively).

17. The composition of claim 1, wherein the CD20 antigen comprises a human CD20 antigen comprising the amino acid sequence of SEQ ID NO:12 or a portion thereof, or wherein the CD38 antigen comprise a human CD38 antigen comprising the amino acid sequence of SEQ ID NO:13.

18. The method of claim 2, wherein the at least one cancer cell is contacted with a therapeutically effective amount of the first and second antibody essentially simultaneously or sequentially in any order.

19. The method of claim 2, wherein the killing of the at least one cancer cell comprises phagocytosis.

20. The method of claim 2, wherein the at least one cancer cell that overexpresses CD47 antigen is selected from a group consisting of an ovarian cancer cell, colon cancer cell, colorectal cancer cell, breast cancer cell and lung cancer cell.

21. The method of claim 2, wherein the at least one cancer cell that overexpresses CD47 antigen is selected from a group consisting of a myeloma, neuroblastic-derived CNS tumor, monocytic leukemia, B-cell derived leukemia, T-cell derived leukemia, B-cell derived lymphoma, T-cell derived lymphoma, non--Hodgkins lymphoma, and mast cell derived tumors.

22. The method of claim 3, wherein the administering comprises administering to the subject
a) the anti-CD47 antibody or antigen binding fragment thereof and the anti-CD20 antibody essentially simultaneously, or the anti-CD47 antibody or antigen binding fragment thereof and the anti-CD20 antibody sequentially in any order; or
b) the anti-CD47 antibody or antigen binding fragment thereof and the anti-CD38 antibody essentially simultaneously, or the anti-CD47 antibody or antigen binding fragment thereof and the anti-CD38 antibody sequentially in any order.

23. The method of claim 3, wherein the administering comprises administering to the subject via a mode selected from a group consisting of intravenous, intramuscular, subcutaneous, intraperitoneal and spinal.

24. The method of claim 3, wherein the cancer that overexpresses CD47 antigen is selected from a group consisting of an ovarian cancer cell, colon cancer cell, colorectal cancer cell, breast cancer cell and lung cancer cell.

25. The method of claim 3, wherein the cancer that overexpresses CD47 antigen is selected from a group consisting of a myeloma, neuroblastic-derived CNS tumor, monocytic leukemia, B-cell derived leukemia, T-cell derived leukemia, B-cell derived lymphoma, T-cell derived lymphoma, non-Hodgkins lymphoma, and mast cell derived tumors.

26. The method of claim 3, wherein the administering comprises administering to the subject the anti-CD47 antibody at a dose of about 20-150 mg/kg or about 30-150 mg/kg or about 40-150 mg/kg or about 50-150 mg/kb or about 60-150 mg/kg or about 70 mg/kg or about 80-150 mg/kg or about 90-150 mg/kg or about 100-150 mg/kg or about 110-150 mg/kg or about 120-150 mg/kg or about 130-150 mg/kg.

27. The method of claim 26, wherein the administering comprises administering to the subject the dose of the anti-CD47 antibody once a week for 2-6 weeks or longer (e.g., up to 10 weeks).

28. The method of claim 3, wherein the administering comprises administering to the subject the anti-CD20 antibody or the anti-CD38 antibody at a suboptimal dose of about 0.1-100 mg/kg.

29. The method of claim 28, wherein the administering comprises administering to the subject the dose of the anti-CD20 antibody or the anti-CD38 antibody once a week for 2-6 weeks or longer (e.g., up to 10 weeks).
